(19) United States
(12) Patent Application Publication
(10) Pub. No.: US 2003/0036066 A1
(43) Pub. Date: Feb. 20, 2003

(54) Linker phosphoramidites for oligonucleotide synthesis

(75) Inventors: Richard T. Pon, Calgary (CA); Shuyan Yu, Calgary (CA)

Correspondence Address: PATENT ADMINSTRATOR, KATTEN MUCHIN ZAVIS ROSENMAN, 525 WEST MONROE STREET, SUITE 1600, CHICAGO, IL, 60661-3693, US

(73) Assignee: University Technologies International Inc.

(21) Appl. No.: 09/948,918

(22) Filed: Sep. 10, 2001

Related U.S. Application Data
(60) Provisional application No. 60/231,301, filed on Sep. 8, 2000.

(51) Int. Cl. C12Q 1/68; C07H 21/04; C07D 473/18; C 07D 4 7/14

(52) U.S. Cl. 435/6; 536/23.1; 544/289; 544/296

(57) ABSTRACT

A novel approach for combining the ease of cleavage of carboxylic acid linker arms with the single phosphoramidite coupling chemistry of the universal supports useful in oligonucleotide synthesis. There is disclosed a new class of phosphoramidite reagents, linker phosphoramidites, which contain a bifunctional linker arm with a protected nucleoside linked through a 3′-ester bond on one end and a reactive phosphoramidite group or other phosphate precursor group on the other end—see FIGS. 2 and 3. The phosphoramidite group on the linker phosphoramidite may be activated under the same conditions and has similar reactivity as conventional nucleoside-3′-phosphoramidite reagents lacking the intermediate linker arm. The 3′-ester linkage contained within the linker phosphoramidite has similar properties to the linkages on prederivatized supports. The ester linkage is stable to all subsequent synthesis steps, but upon treatment with a cleavage reagent, such as ammonium hydroxide, the ester linkage is hydrolyzed. This releases the oligonucleotide product with the desired 3′-hydroxyl terminus and leaves the phosphate portion of the reagent attached to the support, which is subsequently discarded.

### CROSS-REFERENCE TO RELATED APPLICATION

The present application claims the benefit under 35 U.S.C. §119(e) of provisional patent application Ser. No. 60/231,301, filed Sep. 8, 2000, the contents of which are hereby incorporated by reference.

### BACKGROUND OF THE INVENTION

1. Field of the Invention

In one of its aspects the present invention relates to a novel series of phosphorus-containing compounds useful in oligonucleotide synthesis. In another of its aspects, the present invention relates the use of these compounds in oligonucleotide synthesis.

2. Description of the Prior Art

Oligonucleotides have become widely used as reagents for biochemistry and molecular biology (G. M. Blackburn and M. J. Gait, Nucleic Acids in Chemistry and Biology, 1990, IRL Press, Oxford). These materials are used as DNA sequencing primers (C. J. Howe and E. S. Ward, Nucleic Acids Sequencing: A Practical Approach, 1989, IRL Press, Oxford), polymerase chain reaction or “PCR” (N. Smyth Templeton, 1992, Diagnostic Molecular Pathology 1, 58-72) primers, DNA probes (L. J. Kricka, Nonisotopic DNA Probe Techniques, 1992, Academic Press, San Diego) and in the construction of synthetic or modified genes (S. A. Narang, Synthesis and Applications of DNA and RNA, 1987, Academic Press, San Diego). Modified oligonucleotides are also finding widespread use as diagnostic and therapeutic agents—see one or more of:

(a) S. L. Beaucage and R. P. Iyer, 1993, Tetrahedron 49, 6123-6194;

(b) S. L. Beaucage and R. P. Iyer, 1993, Tetrahedron 49, 1925-1963;

(c) S. Verma and F. Eckstein, 1998, Annu. Rev. Biochem. 67, 99-134; and

(d) R. P. Iyer, A. Roland, W. Zhou and K. Ghosh, 1999, Curr. Opin. Molec. Therap. 1, 344-358.

Particularly important has been the development of high density DNA arrays (M. Schena, DNA Microarrays: A Practical Approach, 1999, Oxford University Press, Oxford), which can contain thousands or tens of thousands of different DNA sequences. Consequently, demand for chemically synthesized oligonucleotides has been increasing steadily and many millions of oligonucleotides per year are now required.

Solid-phase chemical synthesis is the only method capable of producing the number of synthetic oligonucleotides required and automated synthesis using phosphoramidite coupling chemistry (S. L. Beaucage and R. P. Iyer, 1992, Tetrahedron 12, 2223-2311) has become the preferred synthetic method. The first step in solid-phase synthesis is attachment of a nucleoside residue to the surface of an insoluble support, such as a controlled pore glass or polystyrene bead, through a covalent linkage (R. T. Pon, “Solid-phase supports for oligonucleotide synthesis”, Unit 3.1 in Current Protocols in Nucleic Acid Chemistry, eds., S. L. Beaucage, D. E. Bergstrom, G. D. Glick and R. A. Jones, 2000, John Wiley & Sons, New York). This linkage must be resistant to all of the chemical steps required to synthesize the oligonucleotide on the surface of the support. Furthermore, the linkage must be cleavable after synthesis is complete to release the oligonucleotide product from the support.

It is also important that the product released from the support have a terminus which is well defined and can participate in subsequent enzymatic reactions, i.e. be recognized by enzymes such as polymerases. The preferred strategies for solid-phase oligonucleotide synthesis all attach the 3′-terminal residue to the support and assemble the oligonucleotide sequence in the 3′- to 5′- direction. After cleavage from the support, a 3′-hydroxyl group is desired since this is identical with the structure created by enzymatic cleavage. A 3′-terminal phosphate is not as satisfactory since this is not extendable by polymerases and such oligonucleotides cannot function as PCR or sequencing primers.

The above linker requirements are satisfied by using a carboxylic or dicarboxylic acid linker arm to attach the first nucleoside residue by means of an ester linkage to the 3′-hydroxyl group. After synthesis, hydrolysis of this ester linkage with ammonium hydroxide releases the oligonucleotide from the support with the desired 3′-OH functionality. Methods for attaching nucleosides to supports by such means are well known, as illustrated by the prior art shown in FIGS. 1-1 and 1-2. In this approach dicarboxylic linker arms such as succinic acid, hydroquinone-O,O′-diacetic acid, diglycolic acid, oxalic acid, malonic acid, etc. are frequently used.

However, the chemistry required to form the carboxylic ester or amide attachments to the supports is different from the phosphoramidite chemistry required to build up the oligonucleotide sequence. Therefore, the nucleoside attachment step is usually done separately from the automated synthesis. The correct prederivatized supports, containing either A, C, G, T or other minor nucleosides, must be selected in advance of automated synthesis. This is satisfactory when producing small numbers of oligonucleotides but becomes tedious and a potential source of error when large numbers of different sequences are synthesized, such as in 96 well plates. Although fast coupling reagents have been developed, which allow automation of the esterification/amidation steps immediately prior to the phosphoramidite synthesis cycles (see R. T. Pon, S. Yu and Y. S. Sanghvi, 1999, Bioconjugate Chemistry 10, 1051-1057 and R. T. Pon and S. Yu, 1999, Synlett, 1778-1780), these reagents require specially modified DNA synthesizers to perform the esterification chemistry as well as the phosphoramidite chemistry.

It is more desirable to have a method which uses only a single coupling chemistry since commercially available automated instrumentation is only designed for phosphoramidite synthesis. A variety of “universal” solid-phase supports containing a diol moiety, which have one hydroxy group free and one hydroxy group either protected or linked to the support, have been developed to meet this need (R. T. Pon, “Solid-phase supports for oligonucleotide synthesis”, Unit 3.1 in Current Protocols in Nucleic Acid Chemistry, eds., S. L. Beaucage, D. E. Bergstrom, G. D. Glick and R.A. Jones, 2000, John Wiley & Sons, New York)—see FIGS. 1-3. In this approach, the same nucleoside-3′-phosphoramidite reagents used to synthesize the oligonucleotide sequence are used to attach the first nucleoside residue to the support. However, this results in the oligonucleotide being attached to the support through a 3′-phosphate and not a 3′-ester linkage. Therefore, cleavage from the support initially produces a 3′-phosphorylated product. Formation of the desired 3′-OH terminus requires either additional reagents or prolonged deprotection time to remove the 3′-phosphate group. The dephosphorylation reaction is also not quantitative and so a mixture of products is produced. Therefore, this approach is unsatisfactory because of the longer processing time, the reduced yield of desired 3′-OH product, and the mixture of 3′-phosphorylated and non-phosphorylated sequences in the final product.

Thus, despite the advances made to date there is still room for improvement. Specifically, it would be desirable to have a new approach to oligonucleotide synthesis which combines the advantages of using phosphoramidite coupling chemistry with the advantages of efficient automated synthesis without the need to resort to the “correct prederivatized supports” referred to above.

### SUMMARY OF THE INVENTION

It is an object of the present invention to obviate or mitigate at least one of the above-mentioned disadvantages of the prior art.

It is an object of the present invention to provide a novel phosphorus-containing compound useful in oligonucleotide synthesis.

It is another object of the present invention to provide a novel process for oligonucleotide synthesis.

Accordingly, in one of its aspects, the present invention provides a compound having Formula I:

X1—Q—Z¹ (I)

wherein:

X¹ comprises a protected nucleoside moiety selected from the following structures:

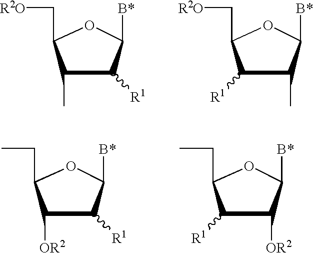

wherein:

R′ is hydrogen, fluorine or —OR³;

R² and R³ are the same or different and each is selected from hydrogen, methyl and a protecting group; and

B* is a nucleic acid base;

Q is a moiety selected from:

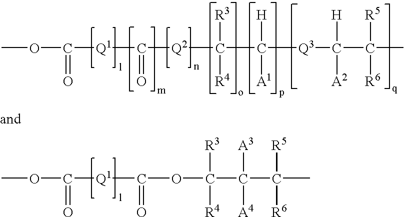

wherein:

Q¹ is an organic moiety;

Q² is selected from —O—, —N(H)—, —N(R⁷)— and —S—;

Q³ is selected from —S(O)₂—, —S(O)—, —C(O)—, —O—, —O—(R⁸)—O— and —R⁹—;

A¹ and A² may be the same or different and each is selected from hydrogen, halogen, a C-₁₋₁₀ alkyl group, a C₅₋₁₀ aryl group, a C₃₋₁₀ cycloalkyl group, —COOR⁷, —CONH, —CONR⁷, —CN, —NO₂, SR⁷, —S(O)R⁷, —S(O)₂R⁷, —SC(C₆H₅)₃, a C₁₋₁₀ alkylsulfonyl group, a C₅₋₁₀ aryl group, a C₁₋₁₀ alkylthio group, —Si(R⁷)₃, a C₁₋₁₀ haloalkyl group, naphthyl, 9-fluorenyl, 2-anthraquinonyl,

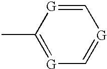

wherein G is C or N with at least one G being N, and

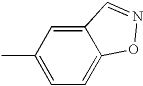

A³ and A⁴ may be the same or different and each is selected from hydrogen, halogen, a C₁₋₁₀ alkyl group, a C₅₋₁₀ aryl group, a C₃₋₁₀ cycloalkyl group and an electron withdrawing group, provided that at least one of A³ and A⁴ comprises an electron withdrawing group;

R³, R⁴, R⁵ and R⁶ are the same or different and each is selected from hydrogen, halogen, a C₁₋₁₀ alkyl group, a C₅₋₁₀ aryl group and a C₃₋₁₀ cycloalkyl group;

R⁷ is selected from a C₁₋₁₀ alkyl group, a C₅₋₁₀ aryl group and a C₃₋₁₀ cycloalkyl group;

R⁸ is a C₁₋₁₀ alkyl group or a C₅₋₁₀ aryl group;

R⁹ is a C₅₋₁₀ aryl group or —CH₂—; and

l, m, n and p are independently 0 or 1;

o is an integer in the range 0-30; and

q is an integer in the range 0-50; and

Z′ is a phosphorylation moiety.

In another of its aspects, the present invention provides a process for producing a compound having Formula I:

X¹—Q—Z¹

wherein:

X¹ comprises a protected nucleoside moiety selected from the following structures:

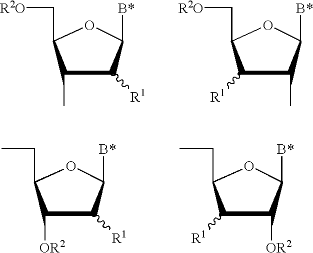

wherein:

R¹ is hydrogen, fluorine or —OR³;

R² and R³ are the same or different and each is selected from hydrogen, methyl and a protecting group; and

B* is a nucleic acid base;

Q is a moiety selected from:

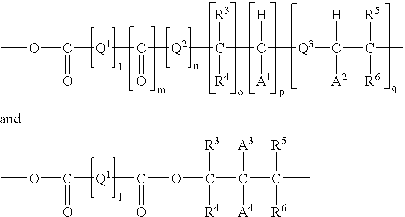

wherein:

Q¹ is an organic moiety;

Q is selected from —O—, —N(H)—, —N(R⁷)— and —S—;

Q³ is selected from —S(O)₂—, —S(O), —C(O)—, —O—, —O—(R⁸)—O— and

A¹ and A² may be the same or different and each is selected from hydrogen, halogen, a C₁₋₁₀ alkyl group, a C_{5-10 aryl group, a C}₃₋₁₀ cycloalkyl group, —COOR⁷, —CONH, —CONR⁷, —CN, —NO₂, —SR⁷, —S(O)R⁷, —S(O)₂R⁷, —SC(C₆H₅)₃, a C₁₋₁₀ alkylsulfonyl group, a C₅o₁₀ aryl group, a C₁₋₁₀ alkylthio group, —Si(R⁷)₃, a C₁₋₁₀ haloalkyl group, naphthyl, 9-fluorenyl, 2-anthraquinonyl,

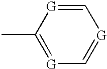

wherein G is C or N with at least one G being N, and

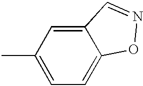

A³ and A⁴ may be the same or different and each is selected from hydrogen, halogen, a C₁₋₁₀ alkyl group, a C₅₋₁₀ aryl group, a C₃₋₁₀ cycloalkyl group and an electron withdrawing group, provided that at least one of A³ and A⁴ comprises and an electron withdrawing group;

R³, R⁴, R⁵ and R⁶ are the same or different and each is selected from hydrogen, halogen, a C₁₋₁₀ alkyl group, a C₅₋₁₀ aryl group and a C₃₋₁₀ cycloalkyl group;

R⁷ is selected from a C₁₋₁₀ alkyl group, a C₅₋₁₀ aryl group and a C₃₋₁₀ cycloalkyl group;

R⁸ is a C₁₋₁₀ alkyl group or a C₅₋₁₀ aryl group;

R⁹ is a C₅₋₁₀ aryl group or —CH₂—; and

l, m, n and p are independently 0 or 1;

o is an integer in the range 0-30; and

q is an integer in the range 0-50; and

Z¹ is a phosphorylation moiety; the process comprising the step of reacting compounds of Formula

II, III and IV:

X¹—OH (II)

H—Q—O—R²⁴ (III)

Z² (IV)

wherein R¹⁸ is a protecting group and Z² is a phosphorus containing precursor to Z¹ or activated phosphorylatoin moiety.

In another of its aspects, the present invention provides a process for producing a derivatized nucleoside having Formula Va or Formula Vb:

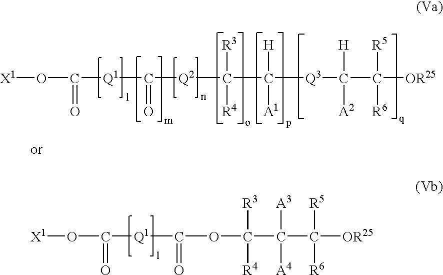

wherein:

X comprises a protected nucleoside moiety selected from the following structures:

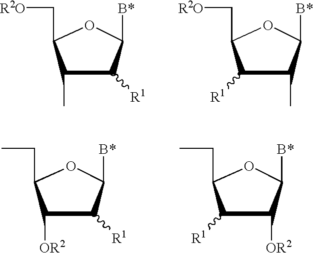

wherein:

R¹ is hydrogen, fluorine or —OR³;

R² and R³ are the same or different and each is selected from hydrogen, methyl and a protecting group; and

B* is a nucleic acid base;

Q¹ is an organic moiety;

Q² is selected from —O—, —N(H)—, —N(R⁷)— and —S—;

Q³ is selected from —S(O)₂—, —S(O)—, —C(O)—, —O—, —O—(R⁸)—O— and —R⁹—;

A¹ and A² may be the same or different and each is selected from hydrogen, halogen, a C₁₋₁₀ alkyl group, a C₅₋₁₀ aryl group, a C₃₋₁₀ cycloalkyl group, —CooR⁷, —CONH, —CONR⁷, —CN, —NO₂, —SR⁷, —S(O)R⁷, —S(O)₂R⁷, —SC(C₆H₅)₃, a C₁₋₁₀ alkylsulfonyl group, a C₅₋₁₀ aryl group, a C₁₋₁₀ alkylthio group, —Si(R⁷)₃, a C₁₋₁₀ haloalkyl group, naphthyl, 9-fluorenyl, 2-anthraquinonyl,

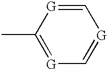

wherein G is C or N with at least one G being N, and

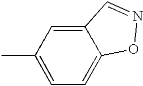

A³ and A⁴ may be the same or different and each is selected from hydrogen, halogen, a C₁₋₁₀ alkyl group, a C₅₋₁₀ aryl group, a C₃₋₁₀ cycloalkyl group and an electron withdrawing group, provided that at least one of A³ and A⁴ comprises an electron withdrawing group;

R³, R⁴, R⁵ and R⁶ are the same or different and each is selected from hydrogen, halogen, a C₁₋₁₀ alkyl group, a C₅₋₁₀ aryl group and a C₃₋₁₀ cycloalkyl group;

R⁷ is selected from a C₁₋₁₀ alkyl group, a C₅₋₁₀ aryl group and a C₃₋₁₀ cycloalkyl group;

R⁸ is a C₁₋₁₀ alkyl group or a C₅₋₁₀ aryl group;

R⁹ is a C₅₋₁₀ aryl group or —CH₂—;

l, m, n and p are independently 0 or 1;

o is an integer in the range 0-30;

q is an integer in the range 0-50; and

R²⁵ is hydrogen or a protecting group;

the process comprising the step of reacting together compounds having Formula II and VI:

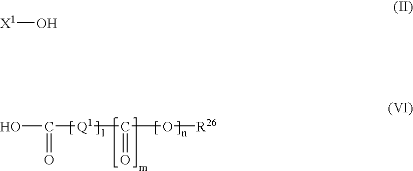

R²⁶ is hydrogen or a protecting group, with a compound having Formula VIIa (in the case where the nucleoside of Formula Va is being produced) or VIIb (in the case where the nucleoside of Formula Vb is being produced):

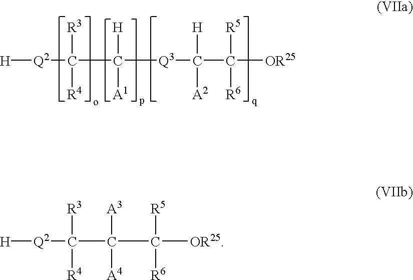

Thus, the present inventors have developed a novel approach for combining the ease of cleavage of carboxylic acid linker arms with the single phosphoramidite coupling chemistry of the universal supports. This entails synthesis of a new class of phosphoramidite reagents, linker phosphoramidites, which contain a bifunctional linker arm with a protected nucleoside linked through a 3′-ester bond on one end and a reactive phosphoramidite group or other phosphate precursor group on the other end—see FIGS. 2 and 3. The phosphoramidite group on the linker phosphoramidite is activated under the same conditions and has similar reactivity as conventional nucleoside-3′-phosphoramidite reagents lacking the intermediate linker arm. The 3′-ester linkage contained within the linker phosphoramidite has similar properties to the linkages on prederivatized supports. The ester linkage is stable to all subsequent synthesis steps, but upon treatment with a cleavage reagent, such as ammonium hydroxide, the ester linkage is hydrolyzed. This releases the oligonucleotide product with the desired 3′-hydroxyl terminus and leaves the phosphate portion of the reagent attached to the support, which is subsequently discarded.

As used throughout this specification, the term “oligonucleotide” is intended to have a broad meaning and encompasses conventional oligonucleotides, backbone-modified oligonucleotides (e.g., phosphorothioate, phosphorodithioate and methyl-phophonate analogs useful as oligotherapeutic agents), labeled oligonucleotides, sugar-modified oligonucleotides and oligonucleotide derivatives such as oligonucleotide-peptide conjugates.

Throughout this specification, when reference is made to a substituted moiety, the nature of the substitution is not specification restricted and may be one or more members selected from the group consisting of hydrogen, a C₁-C₂₀ alkyl group, a C₅-C₃₀ aryl group, a C₅-C₄₀ alkaryl group (each of the foregoing hydrocarbon groups may themselves be substituted with one or more of a halogen, oxygen and sulfur), a halogen, oxygen and sulfur. Further, the term “alkyl”, as used throughout this specification, is intended to encompass hydrocarbon moieties having single bonds, one or more doubles bonds, one or more triples bond and mixtures thereof.

The compound of Formula I is useful in producing oligonucleotides of desired sequence on a support material. In the present specification, the terms “support” and “support material” are used interchangeably and are intended to encompass a conventional solid support. The nature of the solid support is not particularly restricted and is within the purview of a person skilled in the art. Thus, the solid support may be an inorganic substance. Non-limiting examples of suitable inorganic substances may be selected from the group consisting of silica, porous glass, aluminosilicates, borosilicates, metal oxides (e.g., aluminum oxide, iron oxide, nickel oxide) and clay containing one or more of these. Alternatively, the solid support may be an organic substance such as a cross-linked polymer. Non-limiting examples of a suitable cross-linked polymer may be selected from the group consisting of polyamide, polyether, polystyrene and mixtures thereof. One preferred solid support for use herein is conventional and may be selected from controlled pore glass beads and polystyrene beads.

### BRIEF DESCRIPTION OF THE DRAWINGS

Embodiments of the present invention will be described with reference to the accompanying drawings, wherein like numerals designate like elements, and in which:

FIG. 1a illustrates a prior art synthesis of attaching a nucleoside to a support,

FIG. 1b illustrates a prior art approach for synthesizing oligonucleotides in tandem;

FIGS. 2 and 3 illustrate preferred embodiments of the present process;

FIG. 4 illustrates a preferred embodiment of the present process for synthesizing oligonucleotides in tandem;

FIG. 5 illustrates the synthetic routes used in Examples 1-3 below

FIG. 6 illustrates the synthesis of a preferred reagent for tandem synthesis.

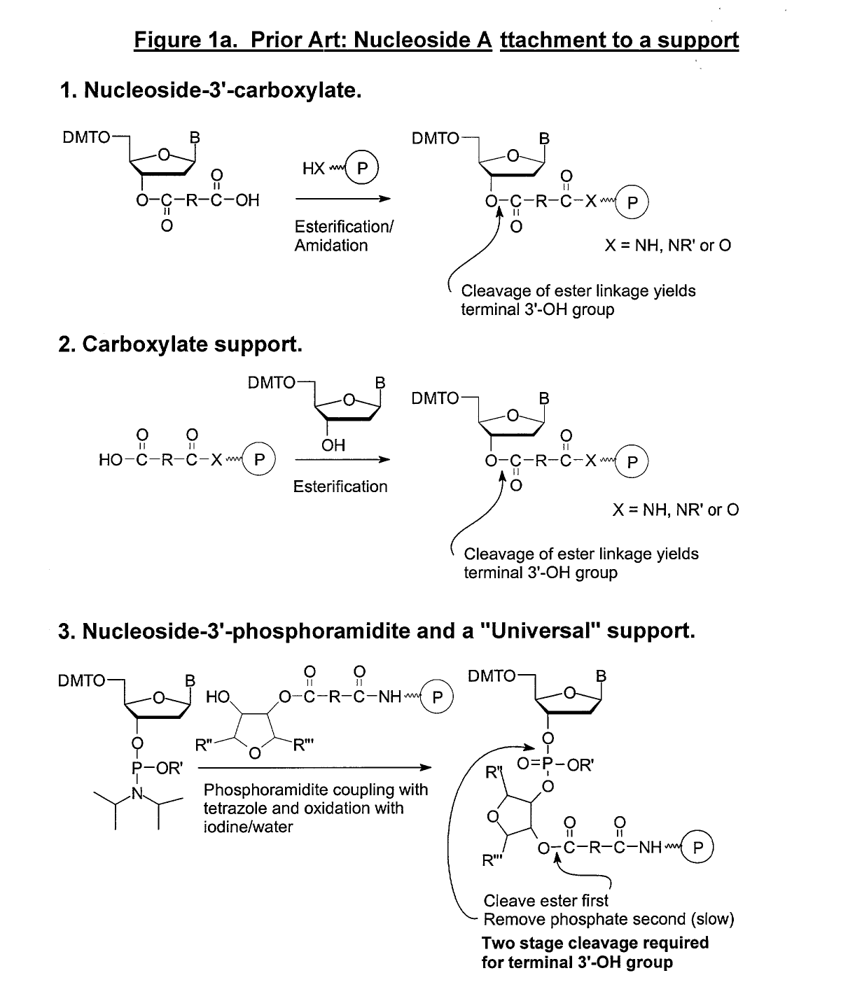

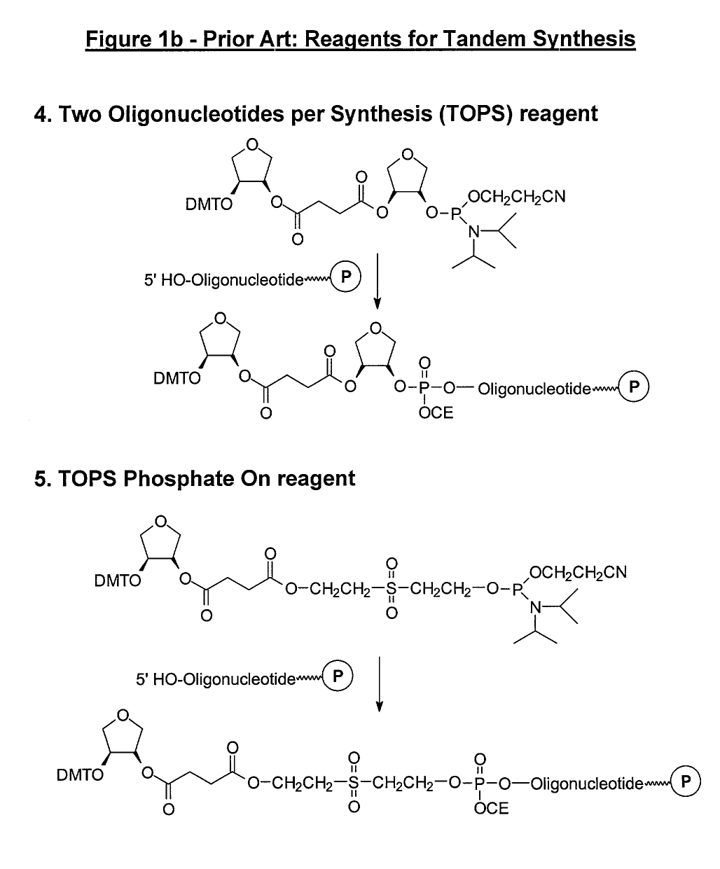

### DETAILED DESCRIPTION OF THE PREFERRED EMBODIMENTS

Phosphoramidite reagents are usually prepared by reacting an alcohol with a trivalent phosphite, such as 2-cyanoethyl diisopropylchlorophosphoramidite, N,N-diisopropylmethyl-phosphonamidic chloride, or bis-(diisopropylamino)-2-cyanoethoxyphosphine. Protected 2′-deoxyribonucleosides, ribonucleosides, or other nucleoside compounds with either free 3′- or 5′-hydroxyl groups are the most common substrates for this reaction since the resulting nucleoside phosphoramidite reagents can be used to assemble oligonucleotide sequences. However, many other reagents such as amino or thiol end-modifiers, non-nucleotide spacers, fluorescent dyes, lipophilic groups such cholesterol or Vitamin E, and non-isotopic labels, such as biotin have also been converted into alcohols and then into phosphoramidite reagents. In these reagents, the phosphoramidite group is used as a reactive group to permanently attach the reagent to the oligonucleotide sequence through a stable phosphate linkage.

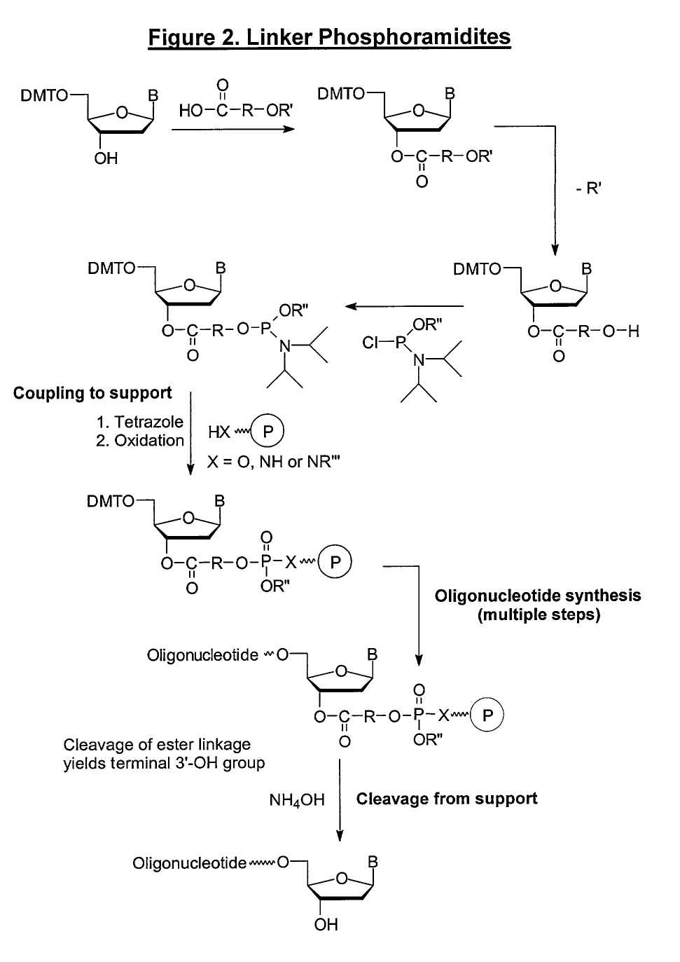

In an aspect of the present invention, a reagent such as a protected nucleoside or a non-nucleoside end modifier with a free hydroxyl group is esterified to a carboxylic acid linker arm. The resulting ester linkage will become the site of subsequent cleavage when exposed to ammonium hydroxide or other cleavage conditions. This internal cleavage site differentiates the linker phosphoramidites of this invention from previous phosphoramidite reagents which never separate the phosphate group from the product. The carboxylic linker arm should have a second site (e.g., hydroxyl) which can react with a trivalent phosphite to convert the reagent into a phosphoramidite reagent. Thus the linker can be any compound with both a carboxylic acid group and an alcohol—see FIG. 2. Examples of possible linkers include, but are not limited to: 4-hydroxymethylphenoxyacetic acid (HMPA); 4-hydroxymethylbenzoic acid (HMBA); 4-(4-hydroxymethyl-3-methoxyphenoxy)-butyric acid (HMPB); 3-(4-hydroxymethylphenoxy)-propionic acid; glycolic acid; lactic acid; 4-hydroxybutyric acid; 3-hydroxybutyric acid; 10-hydroxydecanoic acid; 12-hydroxydodecanoic acid; 16-hydroxyhexadecanoic acid; or 12-hydroxystearic acid.

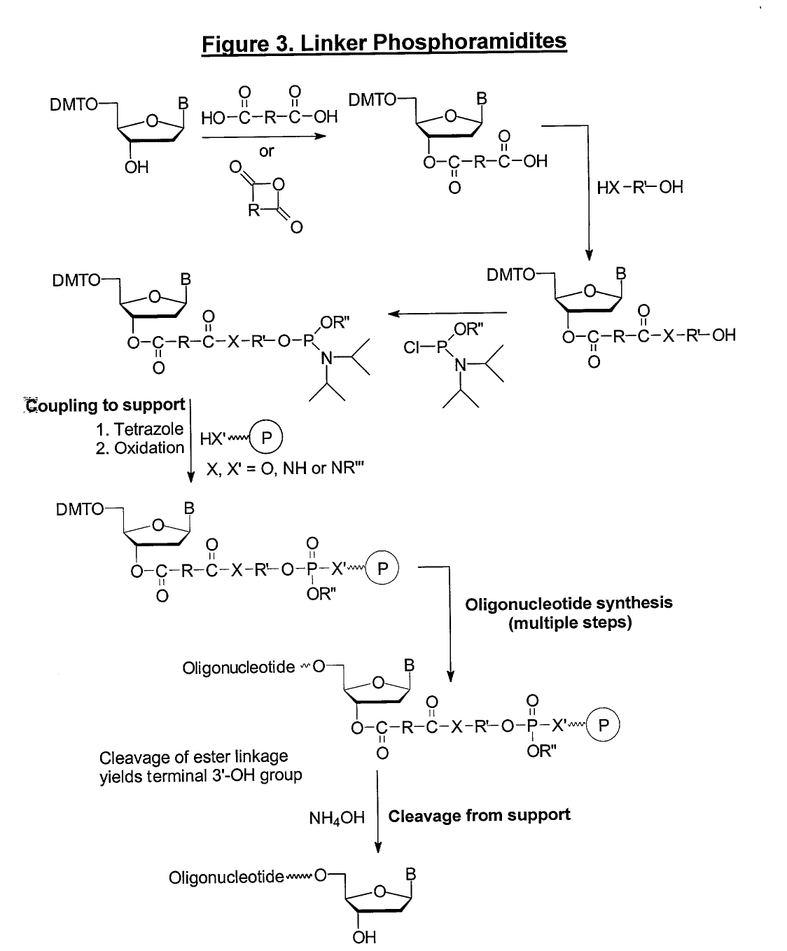

Traditionally, linker arms for solid-phase oligonucleotide synthesis have been dicarboxylic acids such as succinic acid, hydroquinone-O, O′-diacetic acid, diglycolic acid, oxalic acid, malonic acid, etc. and it is desirable to maintain these types of linker arms in the invention because their useful properties have been well established. Therefore, a second route towards synthesis of linker phosphoramidite reagents (FIG. 3) which uses well-known dicarboxylic acids is also possible. In this procedure the cleavable ester linkage is produced by attaching one end of the dicarboxylic acid linker to a nucleoside. The other end of the dicarboxylic acid is then coupled through an ester or amide linkage to a second diol or amino-alcohol which serves to convert the carboxyl group into an alcohol or amino group capable of forming the phosphoramidite portion of the linker phosphoramidite. Examples of possible compounds for the second portion of the linker arm include, but are not limited too: ethylene glycol; diethylene glycol; triethylene glycol; tetraethylene glycol, pentaethylene glycol; hexaethylene glycol; 2-aminoethanol; 1,2-diaminoethane; 1,3-propanediol; 3-amino-1-propanol; 1,3-diaminopropane; 1,4-butanediol; 4-amino-1-butanol; 1,4-diaminobutane; 1,5-pentanediol; 1,6-hexanediol; 6-amino-1-hexanol; 1,6-diaminohexane; or 4-amino-cyclohexanol.

The phosphorus containing group on the end of the linker may be any type of precursor which can be activated and react under oligonucleotide synthesis conditions. A variety of chemistries are known for oligonucleotide synthesis, such as the phosphodiester method, the phosphotriester method, the modified phosphotriester method, the chlorophosphite or phosphite-triester method, the H-phosphonate method, and the phosphoramidite method. However, at the present time, only the last two methods are used regularly and the phosphoramidite method is by the far the most popular.

As used throughout this specification, the term “activation” or “activated phosphorylation moiety” is intended to have broad meaning and refers to the various ways in which a phosphorus group can be attached through either a phosphite ester, phosphate ester, or phosphonate linkage. Phosphorus moieties containing either trivalent (P^{III}) or pentavalent (P^{V}) oxidation states are possible and the oxidation state of the phosphorus may change (usually from P^{III} to P^{V}) during the course of the coupling reactions. Thus, reagents which are precursors to the desired products may have a different oxidation state than the product. The reagents used for phosphorylation may be inherently reactive so that no external activating or coupling reagents are required. Examples of this type include chlorophosphite, chlorophosphate, and imidazole, triazole, or tetrazole substituted phosphite and phosphate reagents. Phosphorylation reagents which are stable until activated by the presence of a separate activating agent are more convenient and are widely used. Examples of these reagent include phosphoramidite and bis-phosphoramidite reagents such as 2-cyanoethyl-N,N′-diisopropylphosphoramidite derivatives and bis-(N,N′-diisopropylamino)-2-cyanoethylphosphine. Reagents with reactive groups may also be substituted with other reactive groups to make for more desirable coupling properties. An example of this is the conversion of highly reactive phosphorus trichloride into phosphorus tris-(imidazolide) or phosphorus tris-(triazolide) species before use. Phosphorylation reagents may also require in situ conversion into activated species by additional coupling reagents. This may be similar to the formation of carboxylic esters and amides where carbodiimide coupling reagents, such as dicyclohexylcarbodiimide or 1-[3-(dimethylamino)propyl]-3-ethylcarbodiimide hydrochloride and similar reagents; uronium coupling reagents, such as O-benzotriazol-1-yl-N,N,N′,N′-tetramethyluronium hexafluorophosphate (HBTU), O-benzotriazol-1-yl-N,N,N′,N′-tetramethyluronium tetrafluoroborate (TBTU) or O-(7-azabenzotriazol-1-yl)-N,N,N′,N′-tetramethyluronium hexafluorophosphate (HATU) and similar reagents; and phosphonium coupling reagents, such as benzotriazol- 1-yloxytris(dimethylamino)phosphonium hexafluorophosphate (BOP) or benzotriazol-1-yloxytripyrrolidinophosphonium hexafluorophosphate (PyBOP) and similar reagents are possible. It may also require coupling reagents which produce mixed anhydride intermediates such as pivaloyl chloride, especially useful for coupling H-phosphonate reagents; and substituted arylsulphonyl chloride, imidazolide, triazolide, and tetrazolide reagents which are especially useful for coupling of phosphate reagents. Phosphorylation reagents may also have protecting groups which allow them to be more easily handled as neutral, uncharged species. These protecting groups are removable to allow the charged species to be produced in situ without isolation and then this charged species participates in the coupling reaction. An example of this approach is known as the modified phosphotriester approach. Thus, there is a broad and diverse range of reagents and reaction conditions for introducing phosphorus groups and for coupling them to produce phosphite, phosphate, and phosphonate linkages. However, these methods are all known to those skilled in the art.

Linker phosphoramidite reagents of the four common bases (A, C, G, and T) or other minor bases can be prepared and installed on automated DNA synthesizers in the same manner as the four conventional nucleoside-3′-phosphoramidite reagents (FIGS. 2 and 3). Inexpensive and readily available underivatized amino or hydroxyl solid-phase supports can then be used as “universal” supports in either column or plate formats. Standard phosphoramidite coupling cycles can then be used to attach the linker phosphoramidite in the first synthesis cycle before switching to conventional phosphoramidite reagents for the subsequent chain extension steps. No additional coupling reagents are required since the activator (usually tetrazole) remains the same for both types of phosphoramidite reagent. Automated synthesizers which can support eight different phosphoramidite reagents at one time are already widely available and so having a set of four linker phosphoramidites and four conventional phosphoramidites installed simultaneously is not a problem. The fact that only four additional linker phosphoramidites are required is a significant advantage over our previous method of automatically attaching the first nucleoside through an ester or amide linkage, since this method required five extra reagents (four nucleosides and a coupling reagent) and synthesizers with this much extra reagent capacity are not readily available.

After completion of the synthesis, cleavage of the product can be performed using the same reagents and conditions as previously used with prederivatized supports and the products will be released with the desired 3′-hydroxyl ends. The phosphate moiety of the linker phosphoramidite will remain attached to the support and is discarded. Depending on the linker arm used in the linker phosphoramidite, the cleavage step can be quite rapid. For example, using a linker phosphoramidite containing hydroquinone-O,O′-diacetic acid, treatment with room temperature ammonium hydroxide for only two minutes is sufficient. Once released from the support, the products must still be deprotected by conventional methods but no further dephosphorylation steps are required and no mixtures of 3′-phosphorylated and 3′-OH products result.

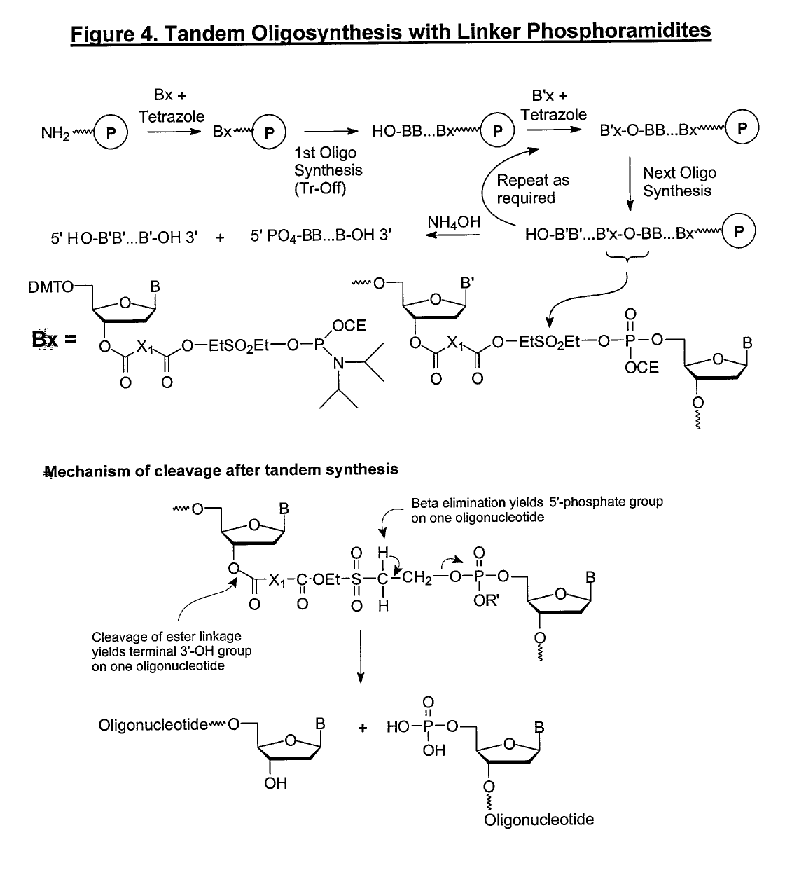

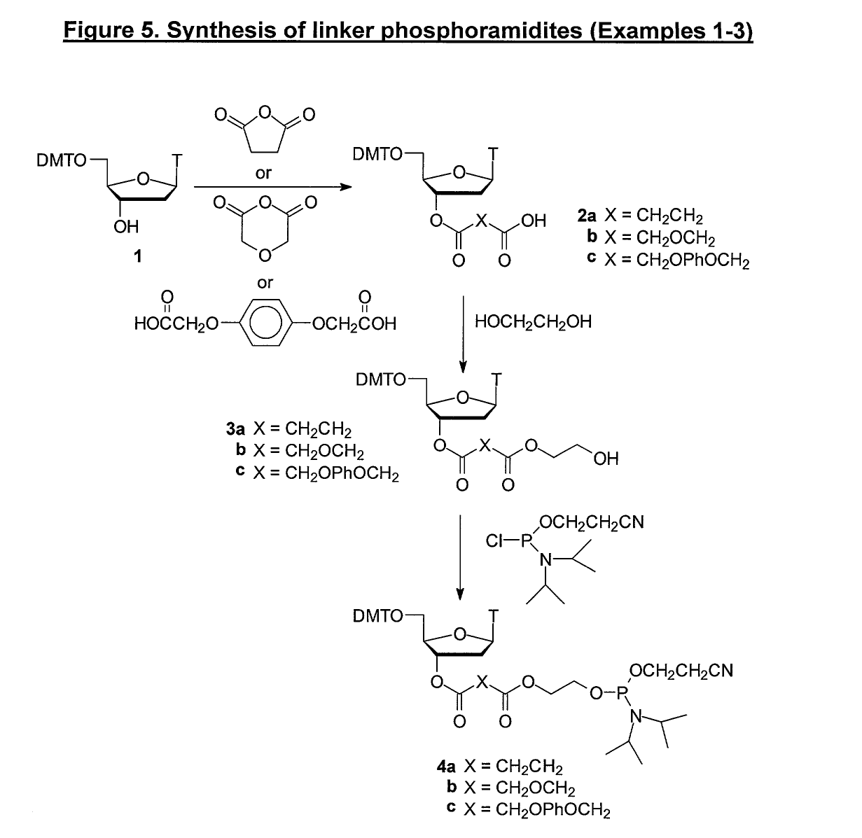

Multiple oligonucleotides can also be produced in tandem on the same synthesis column (FIG. 4). In this process, the first oligonucleotide sequence is synthesized on the support with a 5′-terminal hydroxyl group, i.e., without a 5′-dimethoxytrityl group. The terminal 5′-hydroxyl group of the first oligonucleotide can then serve as a reactive site for a linker phosphoramidite containing the 3′-tenninal base of a second oligonucleotide sequence. This second sequence can be the same or different from the first sequence prepared. After the initial base has been added using a linker phosphoramidite, conventional phosphoramidite reagents are then used to synthesize the remainder of the second sequence. Additional sequences may continue to be built-up on the support until the total number of bases exceeds the pore capacity of the solid-phase support. The multiple oligonucleotides prepared in this fashion preferably are simultaneously released from each other and the surface of the support when treated with the reagent which cleaves the first sequence from the surface of the support. Alternatively, use of different linker phosphoramidites between the oligonucleotide products allows selective and sequential release of the products from the support by adjusting the cleavage conditions for each particular linker phosphoramidite. The phosphate residue from the linker phosphoramidite used to attach the first oligonucleotide sequence to the support may be discarded with the used support. However, the phosphate residue from the subsequent linker phosphoramidite additions will remain attached to the 5′-end of the preceding oligonucleotide. Depending upon the choice of linker phosphoramidite, some residual linker moiety may remain attached to the phosphate residue generating a 5′-terminal phosphodiester group. Although, such 5′-phosphate diester end modifications are not natural, their presence does not interfere with the oligonucleotide's use as a DNA sequencing or PCR primer, which are only sensitive to 3′-end modifications, and so such oligonucleotides can still be used in many applications without serious consequences. A preferred linker phosphoramidite reagent includes a linking group which is eliminated from the 5′-terminal phosphate group under the same conditions as the cleavage. This linker phosphoramidite produces a natural 5′-monophosphate and a natural 3′-OH group on the ends of the preceding oligonucleotide. Oligonucleotides produced using the preferred linker phosphoramidite can participate in both ligation reactions involving the 5′-terminus and primer extension reactions involving the 3′-terminus.

Thus, an aspect of the present invention relates to a compound having Formula I:

X¹—Q—Z¹ (I)

wherein:

X¹ comprises a protected nucleoside moiety selected from the following structures:

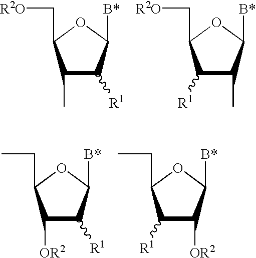

wherein:

R¹ is hydrogen, fluorine or —OR^{3;}

R² and R³ are the same or different and each is selected from hydrogen, methyl and a protecting group; and

B* is a nucleic acid base;

Q is a moiety selected from:

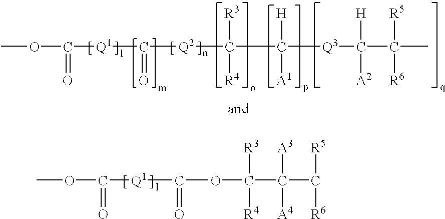

wherein:

Q¹ is an organic moiety;

Q² is selected from —O—, —N(H)—, —N(R⁷)— and —S—;

Q³ is selected from —S(O)₂—, —S(O)—, —C(O), —O—, —OHR⁸)—O— and —R⁹—;

A¹ and A² may be the same or different and each is selected from hydrogen, halogen, a C₁₋₁₀ alkyl group, a C₅₋₁₀ aryl group, a C₃₋₁₀ cycloalkyl group, —COOR⁷, —CONH, —CONR⁷, —CN, —NO₂, —SR⁷, —S(O)R⁷, —S(O)₂R⁷, —SC(C₆H₅)₃, a C₁₋₁₀ alkylsulfonyl group, a C₅₋₁₀ aryl group, a C₁₋₁₀ alkylthio group, —Si(R⁷)₃, a C,-₁₀ haloalkyl group, naphthyl, 9-fluorenyl, 2-anthraquinonyl,

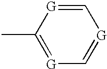

wherein G is C or N with at least one G being N, and

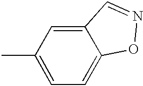

A³ and A⁴may be the same or different and each is selected from hydrogen, halogen, a C₁₋₁₀ alkyl group, a C₅₋₁₀ aryl group, a C₃₋₁₀ cycloalkyl group and an electron withdrawing group, provided that at least one of A³ and A⁴comprises an electron withdrawing group;

R³, R⁴, R⁵ and R⁶ are the same or different and each is selected from hydrogen, halogen, a C₁₋₁₀ alkyl group, a C₅₋₁₀ aryl group and a C₃₋₁ cycloalkyl group;

R⁷ is selected from a C₁₋₁₀ alkyl group, a C₅₋₁₀ aryl group and a C₃₋₁₀ cycloalkyl group;

R⁸ is a C₁₋₁₀ alkyl group or a C₅₋₁₀ aryl group;

R¹⁹ is a C₅₋₁₀ aryl group or —CH₂—;

l, m, n and p are independently 0 or 1;

o is an integer in the range 0-30; and

q is an integer in the range 0-50; and

Z¹ is a phosphorylation moiety.

Preferably, the phosphorylation moiety is selected from the group comprising:

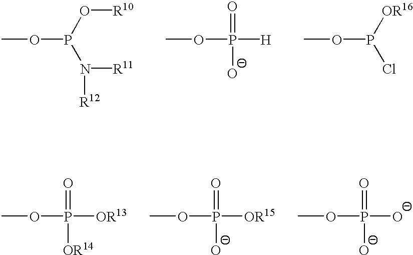

wherein:

R¹¹ and R¹² are the same or different and each may be a substituted or unsubstituted C₁₋₂₀ alkyl group, a substituted or unsubstituted C₅₋₂₀ aryl group, a substituted or unsubstituted C₅₋₂₀ aralkyl group or R¹¹ and R¹² together form a C₃₋₁₀ cycloalkyl group, all of these optionally substituted with one or more heteroatoms selected from oxygen, nitrogen and sulfur; and

R¹⁰, R¹³, R¹⁴, R¹⁵ and R¹⁶ are the same or different and each is a protecting group.

Preferably, the protecting group is selected from the group comprising a substituted or unsubstituted C₁₋₂₀ alkyl group, a substituted or unsubstituted C₅₋₃₀ aryl group, a C₃₋₁₀ cycloalkyl group, a C₅₋₄₀ alkaryl group, a C₁₋₂₀ haloalkyl group, a C₅₋₃₀ haloaryl group, a C₃₋₁₀ halocycloalkyl group, a C₁₋₂₀ nitroalkyl group, a C₅₋₂₀ nitroaryl group, a C₃₋₁₀ nitrocycloalkyl group, a C₁₋₂₀ thioalkyl group, a C₅₋₃₀ thioaryl group, a C₃₋₁₀ thiocycloalkyl group, a C₁₋₂₀ cyanoalkyl group, a C₅₋₃₀ cyanoaryl group, a C₃₋₁₀ cyanocycloalkyl group, a C₁₋₂₀ alkylsilyl group and a C₅₋₃₀ arylsilyl group. More preferably, the protecting group is selected from the group comprising a C₁₋₁₀ alkyl group, a C₅₋₁₀ aryl group, a C₃₋₁₀ cycloalkyl group a C₁₋₁₀ alkylsilyl group, a C₅₋₁₀ arylsilyl group and analogs thereof substituted with one or more of a halogen, oxygen, sulfur, a nitro group, a silyl group, a thio group and a cyano group.

A more preferred phosphorylation moiety is

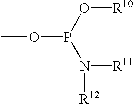

wherein R¹⁰, R¹¹ and R¹² are as defined above. Preferably, R¹⁰, R¹¹ and R¹² are the same or different and each is a C₁₋₁₀ alkyl group, optionally substituted with one or more of a halogen, a nitro group, a thio group and a cyano group. More preferably, R¹¹ and R¹² are the same. Most preferably, each of R¹¹ and R¹² is i-propyl. More preferably, R¹⁰ is a C₁₋₁₀ cyanoalkyl group. Most preferably, R¹⁰ is a cyanoethyl group.

In the compound of Formula I, Q¹ is an organic moiety. Preferably, the organic moiety is a C₁₋₃₀₀ hydrocarbon moiety, optionally substituted with one or more of oxygen, nitrogen, halogen and sulfur.

In one preferred embodiment, Q¹ is selected from the group comprising a C₁₋₄₀ alkyl group, a C₅₋₄₀ aryl group, a C₅₋₄₀ alkyaryl group, a C₃₋₄₀ cycloalkyl group and analogs thereof substituted with one or more of a halogen, oxygen, sulfur, a nitro group, a silyl group, a thio group and a cyano group.

In another preferred embodiment Q¹ has the formula

—CH₂—CH₂—.

In another preferred embodiment, Q¹ has the formula

—CH₂—O—CH₂—.

In yet another preferred embodiment, Q¹ has the formula:

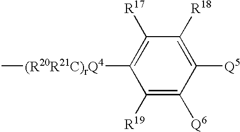

wherein: R¹⁷, R¹⁵ and R¹⁹ are the same or different each is selected from the group comprising hydrogen, halide, a substituted or unsubstituted C₁₋₂₀ alkyl group, a substituted or unsubstituted C₅-C₃₀ aryl group and a substituted or unsubstituted C₅-C₄₀ alkylaryl group; R²⁰ and R²¹ are the same or different and each is selected from the group comprising hydrogen, a halogen, a substituted or unsubstituted C₁-C₂₀ alkyl group, a substituted or unsubstituted C₅-C₃₀ aryl group and a substituted or unsubstituted C₅-C₄₀ alkylaryl group; Q⁴ is selected from the group consisting of —O—, —S—, —C(O), —S(O)₂— and —N(R)—; R is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkylaryl group; r is 0, 1 or 2; and one of Q⁵ and Q⁶ is selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group, and the other of Q⁵ and Q⁶ has the formula:

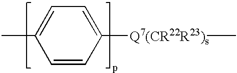

wherein p is 0 or 1, Q⁷ is selected from the group consisting of —O—, —S—, —C(O)—, —S(O)₂— and —N(R)—, R is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkylaryl group, R²² and R²³ are the same or different and are selected from the group consisting of hydrogen, halogen, a substituted or unsubstituted C₁-C₂₀ alkyl group, a substituted or unsubstituted C₅-C₃₀ aryl group and a substituted or unsubstituted C₅-C₄₀ alkylaryl group, and s is 0, 1 or 2.

A highly preferred combination of variables in the compound of Formula I is as follows:

l, m, n, o, p and q are all 1;

Q¹ is selected from

—CH₂—CH₂—

or

—CH— O—CH₂—

or

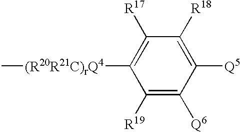

wherein: R¹⁷, R¹⁸ and R¹⁹ are the same or different each is selected from the group comprising hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group; R²⁰ and R²¹ are the same or different and each is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkylaryl group; Q⁴ is selected from the group consisting of —O—, —S—, —C(O)—, —S(O)₂— and —N(R)—; R is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkylaryl group; r is 0, 1 or 2; and one of Q⁵ and Q⁶ is selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group, and the other of Q⁵ and Q⁶ has the formula:

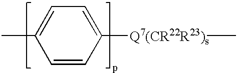

wherein p is 0 or 1, Q⁷ is selected from the group consisting of —O—, —S—, —C(O)—, —S(O)₂— and —N(R)—, R is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkylaryl group, R²² and R²³ are the same or different and are selected from the group consisting of hydrogen, a halogen, a substituted or unsubstituted C₁-C₂₀ alkyl group, a substituted or unsubstituted C₅-C₃₀ aryl group and a substituted or unsubstituted C₅-C₄₀ alkylaryl group, and s is 0, 1 or 2;

Q² is oxygen;

Q³ is —SO₂

A¹, A², A³, R³, R⁴, R⁵, R⁶ are all hydrogen; and

Z¹ has the following structure:

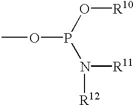

wherein R¹⁰ is 2-cyanoethyl, and R¹¹ and R¹² are each isopropyl.

The compound of Formula may produced by a process comprising the step of reacting together compounds of Formula II, III and IV:

X¹—OH (II)

H—Q—O—R²⁴ (III)

Z² (IV)

wherein R²⁴ is hydrogen or a protecting group and Z² is a phosphorus containing precursor to Z′ or an activated phosphorylatoin moiety.

In one preferred embodiment, R²⁴ is a protecting group and the process comprises the steps of reacting compounds of Formula II and III to produce a reaction product, and thereafter reacting the reaction product with the compound of Formula IV to produce the compound of Formula I.

In another preferred embodiment, R²⁴ is hydrogen and the process comprises the steps of reacting compounds of Formula III and IV to produce a reaction product, and thereafter reacting the reaction product with the compound of Formula II to produce the compound of Formula I.

The use of protecting groups is conventional in the art and the selection thereof is within the purview of a person skilled in the art. Thus, it possible to utilize other protecting groups not specifically referred to in this specification without deviating from the scope of the present invention.

Another aspect of the present invention relates to the use of the compound of Formula I to synthesis one or more oligonucleotides of interest. This is achieved by a process comprising the steps of:

(i) reacting the compound of Formula I with a support material having Formula VIII:

H—X [symbol] {SUPPORT} (IX)

wherein X is selected from —O— and —NR¹⁹—, and R¹⁹ is selected from hydrogen, a C₁₋₁₀ alkyl group, a C₅₋₁₀ aryl group and a C₃₋₁₀ cycloalkyl group to produce a first derivatized support having Formula IX:

X¹—Q—Z¹—X [symbol] {SUPPORT}

(ii) reacting the first derivatized support material of Formula VI with at least one nucleotide until an oligonucleotide sequence corresponding to the first oligonucleotide of interest has been synthesized; and

(iii) cleaving the first oligonucleotide of interest from the compound of Formula IX. As will be appreciated by those of skill in the art, depending on the choice of phosphorylation moiety selected for Z¹, the oxidation state of phosphorus may change from P^{III} to P^{V}.

The other reagents, general reaction conditions and equipment used for oligonucleotide synthesis may be found in the following review articles/textbooks on this topic:

Ilyer et al., Curr. Opin. Molec. Therap., 1999, 1, pgs. 344-358;

Verma et al., Annu. Rev. Biochem., 1998, 67, pgs. 99-134;

Montserra et al., Tetrahedron, 1994, 50, pg. 2617;

Beaucage et al., Tetrahedron, 1993, 49, pgs. 1925-1963;

Beaucage et al., Tetrahedron, 1993, 49, pgs. 6123-6194;

Beaucage et al., Tetrahedron, 1992, 48, pg. 2223;

Davis et al., Innovation and Perspectives in Solid Phase Synthesis (Ed.: R. Epton), Intercept, Andover, 1992, pg. 63;

Englisch et al., Angew. Chemie Intl. Ed. Engl., 1991, pgs. 613-629; and

Goodchild, Bioconjugate Chemistry, 1990, 1, pgs. 165-187.

See, also, one or more of published International patent application WO 97/23497 [Pon et al. (Pon #1)], published International patent application WO 97/23496 [Pon et al. (Pon #2)], published International patent application WO 00/01711 [Pon et al. (Pon #3)] and copending United States patent application Ser. No. ●, filed Sep. 5, 2001 [Pon et al. (Pon #4)].

Embodiments of the present invention will be illustrated with reference to the following Examples which should not be used to limit or construe the scope of the invention.

### EXAMPLE 1

### Synthesis of 5′-dimethoxytritylthymidine-3′-O-(1,2-ethanediol succinate)-(2-cyanoethyl N,N-diisopropyl)-phosphoramidite 4a

5′-Dimethoxytritylthymidine 1 (3.27 g, 6 mmol), succinic anhydride (1.10 g, 10 mmol) and 4-dimethylaminopyridine (147 mg, 1.2 mmol) were dissolved in anhydrous pyridine (40 ml) and stirred at room temperature (2 days). The solution was concentrated by evaporation, redissolved in chloroform and washed with water (2×) and saturated aqueous NaCl. The chloroform solution was dried over magnesium sulfate and evaporated to yield the crude 5′-dimethoxytritylthymidine-3′-O—Succinate 2a (4.50 g), which was used without further purification.

5′-Dimethoxytritylthymidine-3′-O-Succinate 2a (2.84 g, 4.4 mmol) was dissolved in anhydrous acetonitrile (50 ml) and pyridine (2.9 ml) and followed by p-toluenesulfonyl chloride (1.64 g, 8.6 mmol) and N-methylimidazole (1.26 ml, 15.8 mmol). After a clear solution formed, ethylene glycol (0.25 ml, 4.5 mmol) was added and the solution was stirred at room temperature for 20 minutes. The solution was diluted with chloroform, washed consecutively with water, saturated aqueous NaCl, and water. The chloroform solution was concentrated and purified by silica gel chromatography (2% methanol/chloroform) to yield the desired 5′-dimethoxytritylthymidine-3′-O-(1,2-ethanediol succinate) 3a in 31% yield (935 mg). TLC (silica gel, 5% methanol/chloroform) Rf=0.38.

Alternatively, 5′-dimethoxytritylthymidine-3′-O-Succinate 2a (1.29 g, 2 mmol) was dissolved in anhydrous acetonitrile (30 ml) and pyridine (1.3 ml, 16 mmol) and followed by p-toluenesulfonyl chloride (0.74 g, 3.9 mmol) and N-methylimidazole (0.57 ml, 7.2 mmol). After stirring at room temperature (10 min), this solution was added dropwise, via syringe, to ethylene glycol (11.2 ml, 200 mmol). After stirring (30 min), the solvent was concentrated by evaporation, redissolved in chloroform, washed with aqueous sodium bicarbonate and water (2×). The crude product was then purified by silica gel chromatography using 1-2% methanol/chloroform. Yield of 3a, 1.045 g (76%).

The alcohol 3a (923 mg, 1.34 mmol) and diisopropylethylamine (0.91 ml, 5.2 mmol) were dissolved in anhydrous chloroform (8 ml) and 2-cyanoethyl-N,N-diisopropylchlorophosphoramidite (0.39 ml, 1.75 mmol) was added. The reaction was stirred at room temperature for one hour. The reaction was diluted with chloroform, washed with aqueous NaCl (4×) and water and then purified by silica gel chromatography beginning with dichloromethane/hexane/triethylamine 42:53:5 and ending with triethylaminelchloroform 5:95. This yielded the phosphoramidite product 4a in 89% yield (1.06 g). TLC (silica gel, 20% hexane/ethyl acetate) Rf =0.65. 31p NMR (CDCl₃) 5150.754 and 6150.269.

### EXAMPLE 2

### Synthesis of 5′-dimethoxytritylthymidine-3′-O-(1,2-ethanediol diglycolate)-(2-cyanoethyl N,N-diisopropyl)-phosphoramidite 4b

5′-Dimethoxytritylthymidine 1 (1.63 g, 3 mmol), diglycolic anhydride (522 mg, 4.5 mmol) and 4-dimethylaminopyridine (73 mg, 0.6 mmol) were dissolved in anhydrous pyridine (30 ml) and stirred at room temperature (2 days). The solution was concentrated by evaporation, redissolved in chloroform and washed with water (2×), saturated aqueous NaCl and water. The chloroform solution was dried over magnesium sulfate and evaporated to yield the crude 5′-dimethoxytritylthymidine-3′-O-diglycolate 2b (1.93 g, 98%/o), which was used without further purification.

5′-Dimethoxytritylthymidine-3′-O-diglycolate 2b (1.93 g, 2.93 mmol) was dissolved in anhydrous acetonitrile (40 ml) and pyridine (1.9 ml) and followed by p-toluenesulfonyl chloride (1.09 g, 5.7 mmol) and N-methylimidazole (0.84 ml, 10.5 mmol). After a clear solution formed, ethylene glycol (0.16 ml, 2.9 mmol) was added and the solution was stirred at room temperature for 20 minutes. The solution was diluted with chloroform, washed with water and saturated NaCl, concentrated, and purified by silica gel chromatography (2-3% methanol/chloroform) to yield the desired 5′-dimethoxytritylthymidine-3′-O-(1,2-ethanediol diglycolate) 3b in 53% yield (1.09 g). TLC (silica gel, 5% methanol/chloroform) Rf=0.35.

The alcohol 3b (830 mg, 1.18 mmol) and diisopropylethylamine (0.80 ml, 4.6 mmol) were dissolved in anhydrous chloroform (8 ml) and 2-cyanoethyl-N,N-diisopropylchlorophosphoramidite (0.34 ml, 1.5 mmol) was added. The reaction was stirred at room temperature for one hour. The reaction was diluted with chloroform, washed with aqueous NaCl (4×) and water and then purified by silica gel chromatography beginning with dichloromethane/hexane/triethylamine 42:53:5 and ending with triethylamine/chloroform 5:95. This yielded the phosphoramidite product 4b in 67% yield (720 mg). TLC (silica gel, 20% hexane/ethyl acetate) Rf =0.65. ³¹p NMR (CDCl₃) 150.774 and 6150.691.

### EXAMPLE 3

### Synthesis of 5′-dimethoxytritylthymidine-3′-O-(1,2-ethanediol hydroguinone diacetate)-(2-cyanoethyl N,N-diisopropyl)-phosphoramidite 4c

5′-Dimethoxytritylthymidine 1 and hydroquinone-O,O′-diacetic acid were used to prepare 5′-dimethoxytritylthymidine-3′-O-hydroquinone-O, 0 ′diacetate pyridinium or triethylammonium salt 2c as described in Richard T. Pon, “Attachment of Nucleosides to Solid-Phase Supports”, Unit 3.2 in Current Protocols in Nucleic Acids Chemistry, eds. S. L. Beaucage, D. E. Bergstrom, G. D. Glick, and R. A. Jones, John Wiley & Sons, New York, 2000. 2c (2.50 g, 3 mmol) was dissolved in anhydrous acetonitrile (50 ml) and pyridine (1.9 ml). p-Toluenesulfonyl chloride (1.12 g, 5.9 mmol) and N-methylimidazole (0.86 ml, 10.8 mmol) were added. After a clear solution formed, ethylene glycol (0.17 ml, 3.0 mmol) was added and the solution stirred 30 min. The reaction was incomplete and additional ethylene glycol (0.085 ml, 1.5 mmol) was added and the reaction was left overnight. The solution was concentrated by evaporation, diluted with chloroform, and washed with water, saturated aqueous NaHCO₃, and water (2×). The crude product was purified by silica gel chromatography using 0-3% methanol/chloroform to yield 5′-dimethoxytritylthymidine-3′-O-(1,2-ethanediol hydroquinone diacetate) 3c in 35% yield (830 mg).

Alternatively, 2c (2.13 g, 2.5 mmol) was dissolved in anhydrous pyridine (1.6 ml, 20 mmol) and anhydrous acetonitrile (30 ml). p-Toluenesulfonyl chloride (0.93 g, 4.88 mmol) and N-methylimidazole (0.72 ml, 9.0 mmol) were added and the solution was stirred at room temperature (10 min). This solution was then added dropwise, via syringe, with stirring to ethylene glycol (14 ml, 250 mmol). After stirring another 30 min, the reaction was concentrated by evaporation, redissolved in chloroform and washed with saturated aqueous sodium bicarbonate and water (2×). The crude material was purified by silica gel chromatography using a 0-2% (v/v) gradient of methanol in chloroform to yield 3c (719 mg, 36% yield).

Diisopropylethylamine (0.38 ml, 2.2 mmol) and alcohol 3c (382 mg, 0.48 . mmol) were dissolved in anhydrous chloroform (8 ml) and 2-cyanoethyl-N,N-diisopropylchlorophosphoramidite (0.16 ml, 0.72 mmol) was added. After stirring at room temperature for two hours, the reaction was diluted with chloroform, washed with aqueous NaCl (4×) and water and then purified by silica gel chromatography beginning with dichloromethane/hexane/triethylamine 42:53:5 (v/v/v), followed by 5-10% triethylamine in chloroform (v/v). This yielded the phosphoramidite product 4c in 27% yield (128 mg).

### EXAMPLE 4

### Linker Phosphoramidite Rate of Cleavage from CPG Supports

Linker phosphoramidites 4a, 4b, and 4c were dissolved in anhydrous acetonitrile to yield 0.1 M solutions. These solutions were installed on a spare base position of a PE/Biosystems 394 automated DNA synthesizer. A 1 gmole scale synthesis column containing either underivatized long chain alkylamine controlled pore glass (LCAA-CPG) or underivatized glycerol controlled pore glass (Gly-CPG) supports were installed along with the usual tetrazole, deblock, capping, oxidation, and wash reagents for DNA synthesis. A single 1 lmole scale base-addition cycle was then performed to attach the linker phosphoramidite reagents to the CPG supports. The 5′-dimethoxytrityl protecting group was left on the nucleoside in each case.

The synthesis columns were removed from the synthesizer and dried under vacuum (10 min). The CPG supports were removed from the columns, washed again with methanol and chloroform and dried. A weighed aliquot of each support was subjected to dimethoxytrityl analysis to determine the nucleoside loading. A second weighed aliquot was treated with room temperature ammonium hydroxide for a set amount of time. The supports were then washed with water, acetonitrile, methanol, and finally chloroform. The supports were dried and the residual nucleoside loading determined by dimethoxytrityl analysis. The amount of linker cleavage for each linker phosphoramidite is shown in Table 1.

TABLE 1
Cleavage of linker phosphoramidites from CPG supports using room
temperature ammonium hydroxide

| | | Original | Treatment | Residual | |
|---|---|---|---|---|---|
| | | loading | time | loading | Amount of |
| Reagent | Support | (μmol/g) | (min) | (μmol/g) | cleavage |
| 4a | LCAA-CPG | 39 | 60 | 17 | 57% |
| 4a | LCAA-CPG | 39 | 120 | 9 | 77% |
| 4a | Gly-CPG | 54 | 60 | 9 | 83% |
| 4a | Gly-CPG | 54 | 120 | 5 | 91% |
| 4b | LCAA-CPG | 35 | 10 | 4 | 89% |
| 4b | Gly-CPG | 45 | 10 | 3 | 93% |
| 4c | LCAA-CPG | 25 | 2 | 2 | 92% |

The results from Table 1 show the cleavage rates for the linker phosphoramidite reagents are similar to the cleavage rates for nucleosides attached through conventional succinate, diglycolate, or hydroquinone-O, O′-diacetate linker arms.

### EXAMPLE 5

### Oligonucleotide Synthesis of (Tp)₇T Using Linker Phosphoramidite Reagents

The octathymidine sequence, TTTTTTTT, was prepared on an PE/Biosystems 394 DNA synthesizer using standard 1 μmole scale synthesis conditions except the first nucleoside was added using 0.1M linker phosphoramidite reagents 4a-c. Underivatized LCAA-CPG or Gly-CPG supports were used. The initial nucleoside loading was determined by quantitation of the amount of dimethoxytrityl cation released by the first linker phosphoramidite coupling cycle. Overall and average coupling efficiencies were estimated from the first and last trityl colours.

After synthesis, a trityl-off automatic cleavage end procedure was used to release the oligonucleotide product from the support. Sequences produced using 4a-b were cleaved with an automatic 60 min ammonium hydroxide treatment and sequence from 4c were cleaved with an automatic 5 min ammonium hydroxide treatment. The amount of product collected was determined by UV absorption at 260 nm. The results are shown in Table 2.

TABLE 2
Synthesis and cleavage of (Tp)₇T using linker phosphoramidites

| | | Initial | | | | |
|---|---|---|---|---|---|---|
| | | Nucleo- | Overall | Average | | |
| | | side | Coup- | Coup- | Clea- | Amount |
| | | Loading | ling | ling | vage | Recovered |
| | | (μmol/ | Yield* | Yield* | Time | (A₂₆₀ |
| Reagent | Support | g) | (%) | (%) | (min) | units) |
| 4a | LCAA-CPG | 44 | 93.9 | 99.0 | 60 | 57 |
| 4a | Gly-CPG | 57 | 88.6 | 98.0 | 60 | 95 |
| 4b | LCAA-CPG | 45 | 99 | 99.9 | 60 | 67 |
| 4b | Gly-CPG | 58 | 87.9 | 97.9 | 60 | 100 |
| 4c | LCAA-CPG | 20 | 100 | 100 | 5 | 32 |
| 4c | Gly-CPG | 26 | 94.6 | 99.1 | 5 | 46 |

*estimated from trityl analysis.

The (Tp)₇T products prepared from reagents 4a-c on LCAA-CPG support were analyzed by MALDI-TOF mass spectrometry and each oligonucleotide had the expected mass (M+H, calc. 2371.57, observed 2373.0-2374.6). Therefore, the products produced from linker phosphoramidites were identical to the products prepared from conventional synthesis.

### EXAMPLE 6

### Oligonucleotide Synthesis of dGTAAAACGACGGCCAGT Using Linker Phosphoramidite Reagents

The 17 base-long M13 universal priming sequence, dGTAAAACGACGGCCAGT, was prepared on an PE/Biosystems 394 DNA synthesizer using standard 1 tmole scale synthesis conditions except that the first nucleoside was added using 0.1 M linker phosphoramidite reagents 4a-c. Underivatized LCAA-CPG or Gly-CPG supports were used. The initial nucleoside loading was determined by quantitation of the amount of dimethoxytrityl cation released by the first linker phosphoramidite coupling cycle.

After synthesis, a trityl-off automatic cleavage ending procedure was used to release the oligonucleotide product from the support. The amount of product collected was determined by UV absorption at 260 nm. The synthesis supports were then subjected to a second automatic cleavage cycle to determine if any additional material could also be recovered. The results, shown in Table 3, indicate that between 89-94% of the product is released within the first cleavage period.

TABLE 3
Synthesis and cleavage of
dGTAAAACGACGGCCAGT using linker phosphoramidites

| | | Initial | Clea- | 1^{st} Cleavage | 2^{nd} Cleavage |
|---|---|---|---|---|---|
| | | Nucleoside | vage | Amount | Amount |
| Rea- | | Loading | Time | Recovered | Recovered |
| gent | Support | (μmol/g) | (min) | (A₂₆₀ units) | (A₂₆₀ units) |
| 4a | LCAA-CPG | 42 | 60 | 144 | 9 |
| 4a | Gly-CPG | 55 | 60 | 159 | 14 |
| 4b | LCAA-CPG | 38 | 40 | 139 | 9 |
| 4b | Gly-CPG | 54 | 40 | 197 | 20 |
| 4c | LCAA-CPG | 21 | 2 | 85 | 6 |
| 4c | Gly-CPG | 23 | 2 | 75 | 9 |

The M13 primer oligonucleotides prepared from reagents 4a-c on LCAA-CPG support were analyzed by MALDI-TOF mass spectrometry. In each case, the product gave the expected mass (4+H calc. 5228.41, observed 5225.7-5228.2). A control synthesis of the same sequence on a conventional prederivatized LCAA-CPG support was also found to give a similar result by MALDI-TOF mass spectrometry (M+H calc. 5228.41, observed 5229.3). Therefore, the products produced from linker phosphoramidites were identical to the products prepared from conventional synthesis.

### EXAMPLE 7

### Comparison of the Products from Linker Phosphoramidite Synthesis with Products Prepared from Conventional Pre-Derivatized Supports

Samples of the six unpurified octathymidine products prepared in Example 5 and the six 17 base-long M13 universal primer sequences prepared in Example 6 were analyzed by polyacrylamide gel electrophoresis using a 24% polyacrylamide/7M urea gel. Authentic octathymidine and M13 universal primer sequences, synthesized on a conventional long chain alkylamine CPG support prederivatized with 5′-dimethoxytritylthymidine were run along side the above samples for comparison. In addition, octathymidine and M13 universal primer sequences were synthesized with 3′-phosphate and not 3′-hydroxyl groups. These samples were also run alongside the above samples to identify any products which might contain unwanted 3′-phosphate residues. The results show that the linker phosphoramidite products migrate similarly to the authentic products. The 3′-phosphorylated octathymidine marker migrated much faster than any of the linker phosphoramidite products. The 3′-phosphorylated 17 base-long sequence also migrated faster than the non-3′-phosphorylated products, but in this case the difference in mobility was much less.

The above oligonucleotides were also analyzed by capillary gel electrophoresis (CGE) using a Hewlett-Packard 3-D CE instrument, 100 μm×48.5 cm PVA coated capillary, HP replaceable oligonucleotide Polymer A, and HP oligonucleotide buffer. CGE analysis of a mixture of the M13 universal primer sequence made with the 5′-DMT-T-3′—Succinic acid phosphoramidite 4a and a 3′-phosphorylated oligonucleotide with the same sequence showed that the 3′-phosphorylated sequence migrates differently and is completely resolved from the products obtained from the linker phosphoramidites.

Both the polyacrylamide gel and the CGE results showed that products made with the linker phosphoramidites migrated identically with authentic standards, made on prederivatized supports, and differently from the 3′-phosphorylated markers. Therefore, in each case the phosphate residue was being cleaved from the 3′-end of the products as the oligonucleotides were released from the supports.

### EXAMPLE 8

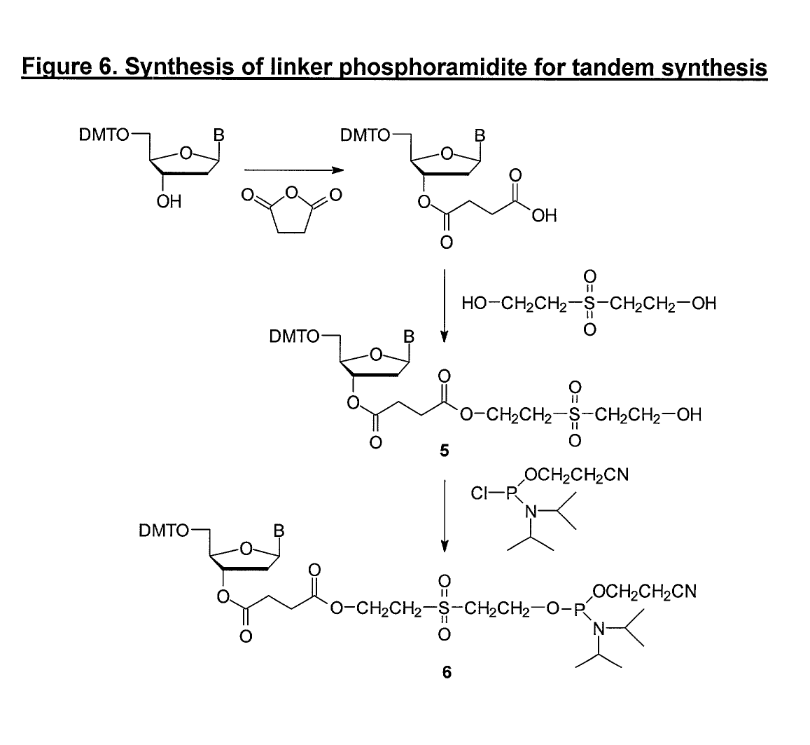

### Synthesis of Linker Phosphoramidites for Tandem Synthesis (FIG. 6)

An aqueous solution of 65% 2,2′-disulphonyldiethanol (10 mmol) was co-evaporated to dryness with anhydrous pyridine (4×20 ml) and the redissolved in anhydrous pyridine (25 ml). 5′-Dimethoxytrityl-N-protected 2′-deoxyribonucleoside-3′-O—Succinic acid hemiester triethylammonium salt (2.0 mmol), 4-dinethylaminopyridine (2.6 mmol), HBTU (2.6 mmol), and diisopropylethylamine (10 mmol) were then added. The reaction was stirred at room temperature (10 min) and TLC (5% methanol/CHCl₃) indicated the reaction was complete. The solution was concentrated by evaporation to remove pyridine, diluted in CHCl₃, washed with water (4×) and evaporated to dryness. The crude product 5 was then purified by silica gel chromatography using 1-3% methanol/CHCl₃. Yields: B=A^{Bz}, 73%; B=C^{Bz}, 80%; B=G^{iBu}, 73%; and B=T, 76%. ESI Mass spectrometry: B=A^{Bz}, M+ Na calc. 917.95, obs. 916; B=C^{Bz}, M+ Na calc. 892.92, obs. 892; B=G^{iBU}, M+ Na calc. 898.93, obs.=898; and B=T, M+ Na calc. 803.85, obs. 803.

Nucleoside 5 (1.28 mmol) was dissolved in a solution of diisopropylethylamine (5.0 mmol) in anhydrous chloroform (15 ml). 2-Cyanoethyl-N,N-diisopropylchlorophosphoramidite (1.66 mmol) was added and the reactions was stirred at room temperature (1 h). The solution was diluted with chloroform and washed with aqueous NaCl (4×). The chloroform solution was concentrated and the product 6 purified by silica gel chromatography using dichloromethane/hexane/triethylamine 42:55:3 to 42:53:5 and then 5% triethylamine/CHCl₃. Yields: B=A^{Bz}, 47%; B=C^{Bz}, 50%; B=G^{iBu}, 49%; and B=T, 56%.

### EXAMPLE 9

### Single Oligonucleotide Synthesis Using Linker Phosphoramidites 6

An ABI 394 DNA synthesizer was configured for synthesis on a 1 μmole scale according to standard methods, except 0.1-0.15M solutions of linker phosphoramidite reagent 6 were installed on spare base positions 5-8. Synthesis columns containing underivatized long chain alkylamine controlled pore glass (LCAA-CPG) containing 102 μmol/g of amino groups were installed in place of prederivatized LCAA-CPG. The synthesizer was then programmed to prepare the sequences shown in Table 4. After synthesis, the products were automatically cleaved from the support using NH₄OH (60 min) and deprotected by heating (55°, 16 h). The crude products were quantitated by UV, coupling yields were estimated from trityl colors, and the sequence identity confirmed by MALDI-TOF mass spectrometry (Table 4).

TABLE 4

Oligonucleotide sequences prepared on underivatized LCAA-CPG using 6.

| | | | | | | |
|---|---|---|---|---|---|---|
| | First | | Average | Crude | | |
| | nucleoside | Overall | Coupling | Product | Calc. | Observe |
| | loading | yield | Yield | (A₂₆₀ | Mass | d Mass |
| Sequence | (μmol/g) | (%) | (%) | units) | (M+H) | (M+H) |

| | | | | | | |
|---|---|---|---|---|---|---|
| dAGCGGATAACAATTTCACA | 41.9 | 74.1 | 98.6 | 167 | 7378.8 | 7370.7 |
| CAGGA | | | | | | |
| dAACTAGTGGATCCCCCGGG | 39.0 | 75.7 | 98.7 | 137 | 7025.5 | 7022.0 |
| CTGC | | | | | | |
| dCGAGGTCGACGGTATCG | 36.1 | 85.7 | 99.0 | 116 | 5251.4 | 5251.7 |
| dGTAAAACGACGGCCAGT | 42.7 | 75.6 | 98.2 | 95 | 5228.4 | 5229.3 |

### EXAMPLE 10

### Synthesis of a 5′-Phosphorylated Oligonucleotide

The 17 base long oligonucleotide sequence with a terminal 5′-phosphate group, 5′-p-dGTAAAACGACGGCCAGT, was prepared as in Example 9, but an additional coupling cycle was performed using reagent 6 (B=T) to add an additional thymidine nucleoside and a 5′-phosphate to the end of the sequence. The sequence was then cleaved from the support and deprotected as in Example 9. During this step the terminal thymidine nucleoside was cleaved from the end of the 17-mer leaving a 5′-phosphate residue. The identical sequence was also synthesized using a conventional “Phosphate On” phosphoramidite reagent to add the terminal 5′-phosphate group. The two products had identical mobility on polyacrylamide gel electrophoresis. MALDI-TOF mass spectrometry was also used to confirm the correct and identical structure of the two oligonucleotides. Oligonucleotide phosphorylated with 6, M+H calc. 5308.4, obs. 5306.1; oligonucleotide phosphorylated with “Phosphate On” reagent, M+H calc. 5308.4, obs 5308.8.

### EXAMPLE 11

### Tandem Synthesis of 5′-Phosphorylated Trinucleotides

A 0.1M solution of linker phosphoramidite 6 (B=T) in acetonitrile was installed on a 394 DNA synthesis on base position #8. A solution of Phosphate On phosphoramidite was installed on position #5. All other reagents were installed as for conventional synthesis. A synthesis column containing 34 mg of 5′-dimethoxytritylthymidine attached to LCAA-CPG through a hydroquinone-O,O′-diacetic acid linker arm was used. The synthesizer was then programmed to prepare the four trinucleotides, d(pAAT), d(pCCT), d(pGGT), and d(pTTT) in one single tandem synthesis by entering the sequence: 5AA8GG8CC8TTT. After synthesis, the products were automatically cleaved from the support using NH40H (60 min) and deprotected (16 h, 55°). Yield: 70.6 A₂₆₀ units.

Linker phosphoramidite solutions of 6 corresponding to the A, G, C, and T nucleosides were respectively installed on positions #5, 6, 7, and 8 on the 394 DNA synthesizer. A synthesis column containing 34.1 mg of 1000 Å low loading LCAA-CPG (10.7 μmol/g) derivatized with 5′-dimethoxytrityl-N-4-benzoyl-2′-deoxycytidine was installed. The synthesizer was then programmed to prepared the following twenty trinucleotide-5′-phosphates, each corresponding to a codon for one amino acid: d(pAAA), d(pAAG), d(pACT), d(pATG), d(pATC), d(pCAC), d(pCAT), d(pCCC), d(CGT), d(pCTC), d(GAA), d(pGAG), d(pGCT), d(pGGT), d(pGTT), d(pTAG), d(pTCT), d(pTGG), d(pTGC), d(pTTC) in one single tandem synthesis by entering the sequence: AA5AA6AC8AT6AT7CA7CA8CC7CG8CT7GA5GA6GC8GG8GT8TA6TC8TG6TG7-TTC. After completion of the above synthesis in the Trityl-ON/Manual mode, the linker phosphoramidite reagent on position #5 was replaced with Phosphate On phosphoramidite and an additional synthesis cycle was run to add a terminal 5′-phosphate group. The products were then automatically cleaved from the support using NH4OH (60 min) and deprotected (16 h, 55°). Yield: 23.6 A₂₆₀ units.

### EXAMPLE 12

### Hydrolysis of the Succinyl Sulfonyldiethanol (Succ-SE) Linker Arm

This Example illustrates the rapid rate with which the sulfonyldiethanol (SE) linker phosphoramidite is hydrolyzed. The cleavage is almost as fast as the cleavage obtained with the linker used in Example 3 hereinabove.

A 0.1 M solution of 5′-dimethoxytritylthymidine-3′-O—Succinyl sulfonyldiethanol phosphoramidite 6 in acetonitrile was installed on a spare base position of an ABI 394 DNA synthesizer. Underivatized LCAA-CPG was used in the synthesis columns. Two syntheses were performed using an otherwise unmodified 1 Fmole scale synthesis cycle.

In the first case only a single phosphoramidite coupling cycle was performed using a trityl-on/manual ending to add the SE linker phosphoramidite to the support. The CPG was removed and the dimethoxytrityl content was determined to be 20.3 μmol/g by quantitative dimethoxytrityl analysis of a portion of the support. Additional portions of the support were then treated with aqueous 28% ammonium hydroxide for periods of 1, 5, and 10 minutes. After washing with methanol and chloroform, dimethoxytrityl analysis of the supports indicated that 92%, 96%, and 98% hydrolysis occurred, respectively, after 1, 5, and 10 minutes.

In the second case, a 21 base long sequence dAGCTAGCTAGCTAGCTAGCTT was prepared using a trityl-off/manual ending. The initial loading of the linker phosphoramidite was determined by dimethoxytrityl analysis to be 20 μmol/g and the average coupling efficiency for the entire synthesis was 99.8%. A special automated ending procedure was then used to deliver portions of aqueous 28% ammonium hydroxide to a collection vial at one minute intervals for a period of 15 minutes. This synthesis produced the oligonucleotide sequence with a free 3′-OH terminus. Each ammonium hydroxide fraction was manually collected, deprotected by heating at 55° overnight, evaporated to remove ammonia, and then quantitated by UV at 260 nm. The cumulative amount of A₂₆₀ units released from the support was then plotted against time to determine the extent of hydrolysis. This experiment indicated that 65%, 94%, and 98% hydrolysis occurred respectively, after 1, 5, and 10 minutes. Thus, cleavage of a 21-base long oligonucleotide sequence from the support is only marginally slower than cleavage of a single nucleoside.

In a third experiment, a commercially available “Phosphate-On” phosphoramidite reagent containing a sulfonyldiethanol linkage was used to phosphorylate a synthesis column containing underivatized LCAA-CPG. The 21-base long sequence DAGCTAGCTAGCTAGCTAGCTT containing a 3′-phosphorylated terminus and not a 3′-OH terminus was then prepared on this support. The initial loading of the Phosphate-On reagent was 33 μmol/g and the average coupling efficiency for the entire synthesis was 99.8%. The rate of hydrolysis in 28% aqueous ammonium hydroxide was then determined as described above. The results indicated that 64%, 98%, and 99% hydrolysis occurred respectively, after 1, 5, and 10 minutes.

These results show that cleavage of the linker phosphoramidite occurs through the elimination of the more labile sulfonyldiethanol function rather than through hydrolysis of the more stable succinic acid linkage. The rate of cleavage observed (98% in 5 min) is almost as fast as the rate of cleavage of the hydroquinone-O,O′-diacetic acid linker arm (98% in˜2 min) and significantly faster than the rate of cleavage of a conventional succinic acid linker arm (98% in 2 h). Thus, sulfonyldiethanol containing linker arms are suitable for applications requiring fast cleavage conditions

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent or patent application was specifically and individually indicated to be incorporated by reference in its entirety.

What is claimed is:

1. A compound having Formula I:
X¹—Q—Z¹ (I)
wherein:
X¹ comprises a protected nucleoside moiety selected from the following structures: 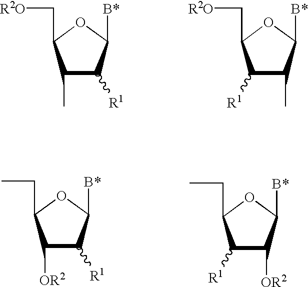
wherein:
R′ is hydrogen, fluorine or —OR³;
R² and R³ are the same or different and each is selected from hydrogen, methyl and a protecting group; and
B* is a nucleic acid base;
Q is a moiety selected from: 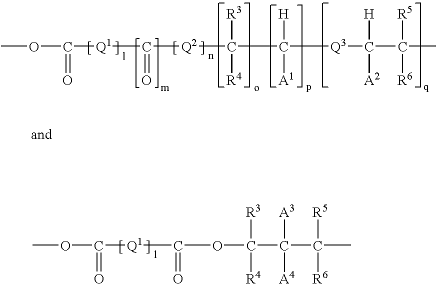
wherein:
Q¹ is an organic moiety;
Q² is selected from −O—, —N(H)—, —N(R⁷) and —S—;
Q³ is selected from —S(O)₂—, —S(O)—, —C(O)—, —O—, —O—(R)—O— and —R⁹—;
A¹ and A² may be the same or different and each is selected from hydrogen, halogen, a C₁₋₁₀ alkyl group, a C₅₋₁₀ aryl group, a C₃₋₁₀ cycloalkyl group, —COOR⁷, —CONH, —CONR⁷, —CN, —NO₂, —SR⁷, —S(O)R⁷, —S(O)₂R⁷, —SC(C₆H₅)₃, a C₁₋₁₀ alkylsulfonyl group, a C₅₋₁₀ aryl group, a C₁₋₁₀ alkylthio group, —Si(R⁷)₃, a C₁₋₁₀ haloalkyl group, naphthyl, 9-fluorenyl, 2-anthraquinonyl, 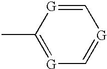
wherein G is C or N with at least one G being N, and 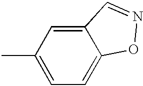
A ³and A⁴ may be the same or different and each is selected from hydrogen, halogen, a C₁₋₁₀ alkyl group, a C₅₋₁₀ aryl grouap, a C₃₋₁₀ cycloalkyl group and an electron withdrawing group, provided that at least one of A³ and A⁴ comprises an electron withdrawing group;
R³, R⁴, R⁵ and R⁶ are the same or different and each is selected from hydrogen, halogen, a C₁₋₁₀ alkyl group, a C₅₋₁₀ aryl group and a C₃₋₁₀ cycloalkyl group;
R⁷ is selected from a C₁₋₁₀ alkyl group, a C₅₋₁₀ aryl group and a C₃₋₁₀ cycloalkyl group;
R⁹ is a C₁₋₁₀ alkyl group or a C₅₋₁₀ aryl group;
R⁹ is a C₅₋₁₀ aryl group or —CH₂—;
l, m, n and p are independently 0 or 1;
o is an integer in the range 0-30; and
q is an integer in the range 0-50; and
Z¹ is a phosphorylation moiety.

2. The compound defined in claim 1, wherein the phosphorylation moiety is selected from the group comprising: 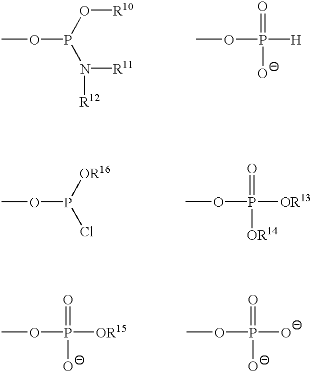
wherein:
R¹¹ and R¹² are the same or different and each may be a substituted or unsubstituted C₁₋₂₀ alkyl group, a substituted or unsubstituted C₅₋₂₀ aryl group, a substituted or unsubstituted C₅₋₂₀ aralkyl group or R¹¹ and R¹² together form a C₃₋₁₀ cycloalkyl group, all of these optionally substituted with one or more heteroatoms selected from oxygen, nitrogen and sulfur;
and R¹⁰, R¹³, R¹⁴, R¹⁵ andR¹⁶ are the same or different and each is a protecting group.

3. The compound defined in claim 2, wherein the protecting group is selected from the group comprising a substituted or unsubstituted C₁₋₂₀ alkyl group, a substituted or unsubstituted C₅₋₃₀ aryl group, a C₃₋₁₀ cycloalkyl group, a C₅₋₄₀ alkaryl group, a C₁₋₂₀ haloalkyl group, a C₅₋₃₀ haloaryl group, a C₃₋₁₀ halocycloalkyl group, a C₁₋₂₀ nitroalkyl group, a C₅₋₂₀ nitroaryl group, a C₃₋₁₀ nitrocycloalkyl group, a C₁₋₂₀ thioalkyl group, a C₅₋₃₀ thioaryl group, a C₃₋₁₀ thiocycloalkyl group, a C₁₋₂₀ cyanoalkyl group, a C₅₋₃₀ cyanoaryl group, a C₃₋₁₀ cyanocycloalkyl group, a C₁₋₂₀ alkylsilyl group and a C₅₋₃₀ arylsilyl group.

4. The compound defined in claim 2, wherein the protecting group is selected from the group comprising a C₁₋₁₀ alkyl group, a C₅₋₁₀ aryl group, a C₃₋₁₀ cycloalkyl group, a C₁₋₁₀ alkylsilyl group, a C₅₋₁₀ arylsilyl group and analogs thereof substituted with one or more of a halogen, oxygen, sulfur, a nitro group, a silyl group, a thio group and a cyano group.

5. The compound defined in claim 1, wherein the phosphorylation moiety is 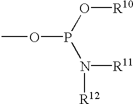
wherein R¹⁰, R¹¹ and R¹² are as defined above.

6. The compound defined in claim 5, wherein R¹⁰, R¹¹ and R¹² are the same or different and each is a C₁₋₁₀ alkyl group, optionally substituted with one or more of a halogen, a nitro group, a thio group and a cyano group.

7. The compound defined in claim 5, wherein R¹¹ and R¹² are the same.

8. The compound defined in claim 5, wherein each of R¹¹ and R¹² is i-propyl.

9. The compound defined in claim 5, wherein R¹⁰ is a C₁₋₁₀ cyanoalkyl group.

10. The compound defined in claim 5, wherein R¹⁰ is a cyanoethyl group.

11. The compound defined in claim 1, wherein Q¹ is selected from the group comprising a C₁₋₄₀ alkyl group, a C₅₋₄₀ aryl group, a C₅₋₄₀ alkyayl group, a C₃₋₄₀ cycloalkyl group and analogs thereof substituted with one or more of a halogen, oxygen, sulfur, a nitro group, a silyl group, a thio group and a cyano group.

12. The compound defined in claim 1, wherein Q¹ has the formula
—CH₂—CH₂—.

13. The compound defined in claim 1, wherein Q¹ has the formula
—CH₂—O—CH₂—.

14. The compound defined in claim 1, wherein Q¹ has the formula: 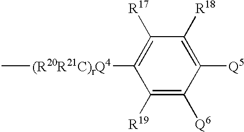
wherein: R¹⁷, R¹⁸ and R¹⁹ are the same or different each is selected from the group comprising hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group; R²⁰ and R²¹ are the same or different and each is selected from the group comprising hydrogen, a halogen, a substituted or unsubstituted C₁-C₂₀ alkyl group, a substituted or unsubstituted C₅-C₃₀ aryl group and a substituted or unsubstituted C₅-C₄₀ alkylaryl group; Q⁴ is selected from the group consisting of —O—, —S—, —C(O)—, —S(O)₂— and —N(R)—; R is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkylaryl group; r is 0, 1 or 2; and one of Q⁵ and Q⁶ is selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group, and the other of Q⁵ and Q⁶ has the formula: 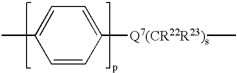
wherein p is 0 or 1, Q⁷ is selected from the group consisting of —O—, —S—, —C(O)—, —S(O)₂— and —N(R)—, R is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkylaryl group, R²² and R²³ are the same or different and are selected from the group consisting of hydrogen, halogen, a substituted or unsubstituted C₁-C₂₀ alkyl group, a substituted or unsubstituted C₅-C₃₀ aryl group and a substituted or unsubstituted C₅-C₄₀ alkylaryl group, and s is 0, 1 or 2.

15. The compound defined in claim 1, wherein:
l, m, n, o, p and q are all 1;
Q¹ is selected from
—CH₂—CH₂—
or
—CH₂—O—CH₂—
or 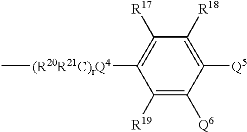
wherein: R¹⁷, R¹⁵ and R¹⁹ are the same or different each is selected from the group comprising hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group; R²⁰ and R²¹ are the same or different and each is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkylaryl group; Q⁴ is selected from the group consisting of —O—, —S—, —C(O)—, —S(O)₂— and —N(R)—; R is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkylaryl group; r is 0, 1 or 2; and one of Q⁵ and Q⁶ is selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group, and the other of Q⁵ and Q⁶ has the formula: 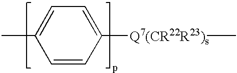
wherein p is 0 or 1, Q⁷ is selected from the group consisting of —O—, —S—, —C(O)—, —S(O)₂— and —N(R)—, R is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkylaryl group, R²² and R²³ are the same or different and are selected from the group consisting of hydrogen, a halogen, a substituted or unsubstituted C₁-C₂₀ alkyl group, a substituted or unsubstituted C₅-C₃₀ aryl group and a substituted or unsubstituted C₅-C₄₀ alkylaryl group, and s is 0, 1 or 2;
Q2 is oxygen;
Q′ is—SO₂
A¹, A², R³, R⁴, R⁵, R⁶ are all hydrogen; and
Z¹ has the following structure: 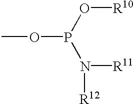
wherein R¹⁰ is 2-cyanoethyl, and R¹¹ and R¹² are each isopropyl.

16. A process for producing a compound having Formula I:
X¹—Q—Z¹ (I)
wherein:
X¹ comprises a protected nucleoside moiety selected from the following structures: 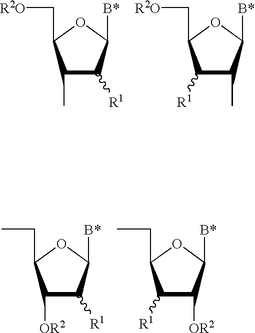
wherein:
R¹ is hydrogen, fluorine or —OR³;
R² and R³ are the same or different and each is selected from hydrogen, methyl and a protecting group; and
B* is a nucleic acid base;
Q is a moiety selected from: 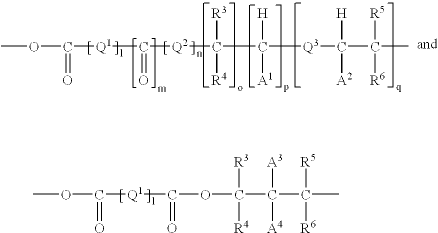
wherein:
Q¹ is an organic moiety;
Q² is selected from —O—, —N(H)—, —N(R⁷)— and —S—;
Q³ is selected from —S(O)₂—, —S(O)—, —C(O)—, —O—, —O—(R⁸)—O— and A¹ and A² may be the same or different and each is selected from hydrogen, halogen, a C₁₋₁₀ alkyl group, a C₅-1₀ aryl group, a C₃₋₁₀ cycloalkyl group, —COOR⁷, —CONH, —CONR⁷, —CN, -NO2, —SR⁷, —S(O)R⁷, —S(0)₂R⁷, —SC(C₆H₅)₃, a Clo₁₀ alkylsulfonyl group, a C₅₋₁₀ aryl group, a C₁₋₁₀ alkylthio group, —Si(R⁷)₃, a C₁₋₁₀ haloalkyl group, naphthyl, 9-fluorenyl, 2-anthraquinonyl, 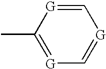
wherein G is C or N with at least one G being N, and 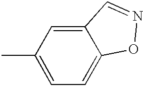
A³ and A⁴ may be the same or different and each is selected from hydrogen, halogen, a C₁₋₁₀ alkyl group, a C₅₋₁₀ aryl group, a C₃₋₁₀ cycloalkyl group and an electron withdrawing group, provided that at least one of A³ and A⁴ comprises and an electron withdrawing group;
R³, R⁴, R⁵ and R⁶ are the same or different and each is selected from hydrogen, halogen, a C₁₋₁₀ alkyl group, a C₅₋₁₀ aryl group and a C₃₋₁₀ cycloalkyl group;
R⁷ is selected from a C₁₋₁₀ alkyl group, a C₅₋₁₀ aryl group and a C₃₋₁₀ cycloalkyl group;
R⁸ is a C₁₋₁₀ alkyl group or a C₅₋₁₀ aryl group;
R⁹ is a C₅₋₁₀ aryl group or —CH₂—;
l, m, n and p are independently 0 or 1;
o is an integer in the range 0-30; and
q is an integer in the range 0-50; and
Z¹ is a phosphorylation moiety;
the process comprising the step of reacting compounds of Formula II, III and IV:
X¹—OH (II)
H—Q—O—R²⁴ (III)
Z² (IV)
wherein R²⁴ is hydrogen or a protecting group and Z² is a phosphorus containing precursor to Z¹ or an activated phosphorylatoin moiety.

17. The process defined in claim 16, wherein r is a protecting group and the process comprises the steps of reacting compounds of Formula II and III to produce a reaction product, and thereafter reacting the reaction product with the compound of Formula IV to produce the compound of Formula I.

18. The process defined in claim 16, wherein R²⁴ is hydrogen and the process comprises the steps of reacting compounds of Formula III and IV to produce a reaction product, and thereafter reacting the reaction product with the compound of Formula II to produce the compound of Formula I.

19. The process defined in claim 16, wherein Z¹ is selected from the group comprising: 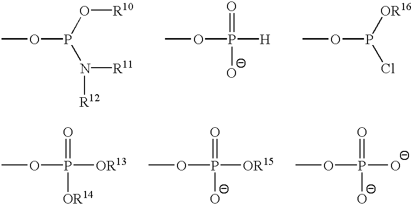
wherein:
R¹¹ and R¹² are the same or different and each may be a substituted or unsubstituted C₁₋₂₀ alkyl group, a substituted or unsubstituted C₅₋₂₀ aryl group, a substituted or unsubstituted C₅₋₂₀ aralkyl group or R¹¹ and R¹² together form a C₃₋₁₀ cycloalkyl group, all of these optionally substituted with one or more heteroatoms selected from oxygen, nitrogen and sulfur; and
and R¹⁰, R¹³, R¹⁴m R¹⁵ and R¹⁶ are the same or different and each is a protecting group.

20. The process defined in claim 19, wherein the protecting group is selected from the group comprising a substituted or unsubstituted C₁₋₂₀ alkyl group, a substituted or unsubstituted C₅₋₃₀ aryl group, a C₃₋₁₀ cycloalkyl group, a C₅₋₄₀ alkaryl group, a C₁₋₂₀ haloalkyl group, a C₅₋₃₀ haloaryl group, a C₃₋₁₀ halocycloalkyl group, a C₁₋₂₀ nitroalkyl group, a C₅₋₂₀ nitroaryl group, a C₃₋₁o nitrocycloalkyl group, a Cl ₂₀ thioalkyl group, a C₅₋₃₀ thioaryl group, a C₃₋₁₀ thiocycloalkyl group, a C₁₋₂₀ cyanoalkyl group, a C₅₋₃₀ cyanoaryl group, a C₃₋₁₀ cyanocycloalkyl group, a C₁₋₂₀ alkylsilyl group and a C₅₋₃₀ arylsilyl group.

21. The process defined in claim 19, wherein the protecting group is selected from the group comprising a C₁₋₁₀ alkyl group, a C₅₋₁₀ aryl group, a C₃₋₁₀ cycloalkyl group, a C₁₋₁₀ alkylsilyl group, a C₅₋₁₀ arylsilyl group and analogs thereof substituted with one or more of a halogen, oxygen, sulfur, a nitro group, a silyl group, a thio group and a cyano group.

22. The process defined in claim 16, wherein Z′ is 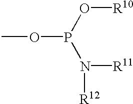
wherein R¹⁰, R¹¹ and R¹² are as defined above.

23. The process defined in claim 22, wherein R¹⁰, R¹¹ and R¹² are the same or different and each is a C₁₋₁₀ alkyl group, optionally substituted with one or more of a halogen, oxygen, sulfur, a nitro group, a silyl group, a thio group and a cyano group.

24. The process defined in claim 22, wherein R¹¹ and R¹² are the same.

25. The process defined in claim 22, wherein each of R¹ and R² is i-propyl.

26. The process defined in claim 22, wherein R¹⁰ is a C₁₋₁₀ cyanoalkyl group.

27. The process defined in claim 22, wherein R¹⁰ is a cyanoethyl group.

28. The process defined in claim 16, wherein Q¹ is selected from the group comprising a C₁₋₄₀ alkyl group, a C₅₋₄₀ aryl group, a C₅₋₄₀ alkylaryl group, a C₃₋₄₀ cycloalkyl group and analogs thereof substituted with one or more of a halogen, oxygen, sulfur, a nitro group, a silyl group, a thio group and a cyano group.

29. The process defined in claim 16, wherein Q¹ has the formula
—CH₂—CH₂—.

30. The process defined in claim 16, wherein Q¹ has the formula
13 CH₂—O—CH₂—.

31. The process defined in claim 16, wherein Q¹ has the formula: 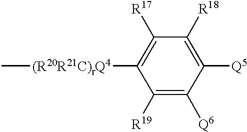
wherein: R¹⁷, R¹⁸ and R¹⁹ are the same or different each is selected from the group comprising hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group; R²⁰ and R²¹ are the same or different and each is selected from the group comprising hydrogen, a halogen, a substituted or unsubstituted C₁-C₂₀ alkyl group, a substituted or unsubstituted C₅-C₃₀ aryl group and a substituted or unsubstituted C₅-C₄₀ alkylaryl group; Q⁴ is selected from the group consisting of —O—, —S—, —C(O)—, —S(O)₂— and —N(R)—; R is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkylaryl group; r is 0, 1 or 2; and one of Q⁵ and Q⁶ is selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group, and the other of Q⁵ and Q⁶ has the formula: 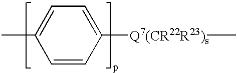
wherein p is 0 or 1, Q⁷ is selected from the group consisting of —O—, —S—, —C(O)Δ, —S(O)₂— and —N(R)—, R is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkylaryl group, R²² and R²³ are the same or different and are selected from the group consisting of hydrogen, a halogen, a substituted or unsubstituted C₁-C₂₀ alkyl group, a substituted or unsubstituted C₅-C₃₀ aryl group and a substituted or unsubstituted C₅-C₄₀ alkylaryl group, and s is 0, 1 or 2.

32. The process defined in claim 16, wherein:
l, m, n, o, p and q are all 1;
Q¹ is selected from
—CH₂—CH₂—
or
—CH₂—O—CH₂—
or 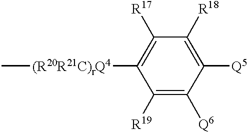
wherein: R¹⁷, R¹⁸ and R¹⁹ are the same or different each is selected from the group comprising hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group; R²⁰ and R²¹ are the same or different and each is selected from the group comprising hydrogen, a halogen, a substituted or unsubstituted C₁-C₂₀ alkyl group, a substituted or unsubstituted C₅-C₃₀ aryl group and a substituted or unsubstituted C₅-C₄₀ alkylaryl group; Q⁴ is selected from the group consisting of —O—, —S—, —C(O)—, —S(O)— and —N(R)—; R is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkylaryl group; r is 0, 1 or 2; and one of Q⁵ and Q⁶ is selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group, and the other of Q⁵ and Q⁶ has the formula: 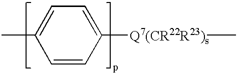
wherein p is 0 or 1, Q⁷ is selected from the group consisting of —O—, —S—, —C(O)—, —S(O)₂— and —N(R)—, R is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkylaryl group, R²² and R²³ are the same or different and are selected from the group consisting of hydrogen, a halogen, a substituted or unsubstituted C₁-C₂₀ alkyl group, a substituted or unsubstituted C₅-C₃₀ aryl group and a substituted or unsubstituted C₅-C₄₀ alkylaryl group, and s is 0, 1 or 2;
Q² is oxygen;
Q³is —SO₂
A¹, A², R³, R⁴, R⁵, R⁶ are all hydrogen; and
Z¹ has the following structure: 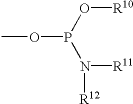
wherein R¹⁰ is 2-cyanoethyl, and R¹¹ and R¹² are each isopropyl.

33. A process for producing a first oligonucleotide of interest comprising the steps of:
(i) reacting a compound of Formula I:
X¹—Q—Z¹ (I)
wherein:
X¹ comprises a protected nucleoside moiety selected from the following structures: 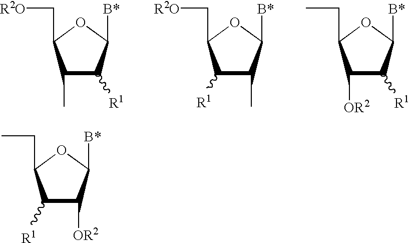
wherein:
R¹ is hydrogen, fluorine or —OR^{3;}
R² and R³ are the same or different and each is selected from hydrogen, methyl and a protecting group; and
B* is a nucleic acid base;
Q is a moiety selected from: 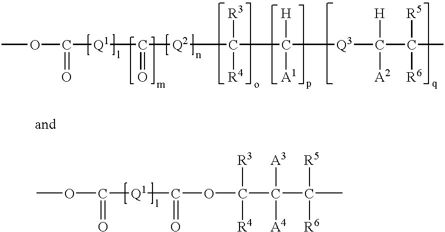
wherein:
Q¹ is an organic moiety;
Q² is selected from —O—, —N(H)—, —N(R⁷) and —S—;
Q³ is selected from —S(O)₂—, —S(O)—, —C(O)—, —O—, —O—(R⁸)—O and —R⁹—;
A¹ and A² may be the same or different and each is selected from hydrogen, halogen, a C₁₋₁₀ alkyl group, a C₅₋₁₀ aryl group, a C₃₋₁₀ cycloalkyl group, —COOR⁷, —CONH, —CONR⁷, —N, —NO₂, —SR⁷, —S(O)R⁷, —S(O)₂R⁷, —SC(C₆H₅)₃, a C₁₋₁₀ alkylsulfonyl group, a C₅₋₁₀ aryl group, a C₁₋₁₀ alkylthio group, —Si(R⁷)₃, a C₁₋₁₀ haloalkyl group, naphthyl, 9-fluorenyl, 2-anthraquinonyl, 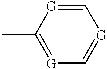
wherein G is C or N with at least one G being N, and 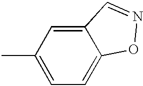
A³ and A⁴ may be the same or different and each is selected from hydrogen, halogen, a C₁₋₁₀ alkyl group, a C₅₋₁₀ aryl group, a C₃₋₁₀ cycloalkyl group and an electron withdrawing group, provided that at least one of A³ and A⁴ comprises an electron withdrawing group;
R³, R⁴, R⁵ and R⁶ are the same or different and each is selected from hydrogen, halogen, a C₁₋₁₀ alkyl group, a C₅₋₁₀ aryl group and a C₃₋₁₀ cycloalkyl group;
R⁷ is selected from a C₁₋₁₀ alkyl group, a C₅₋₁₀ aryl group and a C₃₋₁₀ cycloalkyl group;
R⁸ is a C₁₋₁₀ alkyl group or a C₅₋₁₀ aryl group;
R⁹ is a C₅₋₁₀ aryl group or —CH₂; —l, m, n and p are independently 0 or 1;
o is an integer in the range 0-30; and
q is an integer in the range 0-50; and
Z¹ is a phosphorylation moiety,
with a support material having Formula VIII:
H—X [symbol] {SUPPORT} (VIII)
wherein X is selected from —O— and —NR¹⁹—, and R¹⁹ is selected from hydrogen, a C₁₋₁₀ alkyl group, a C₅₋₁₀ aryl group and a C₃₋₁₀ cycloalkyl group to produce a first derivatized support having Formula IX:
X¹—Q—Z¹ [symbol] {SUPPORT} (IX)
(ii) reacting the first derivatized support material of Formula VI with at least one nucleotide until an oligonucleotide sequence corresponding to the first oligonucleotide of interest has been synthesized; and
(iii) cleaving the first oligonucleotide of interest from the compound of Formula IX.

34. The process defined in claim 33, wherein the phosphorylation moiety is selected from the group comprising: 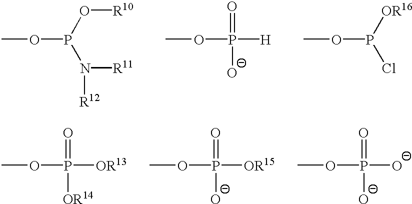
wherein:
R¹¹ and R¹² are the same or different and each may be a substituted or unsubstituted C₁₋₂₀ alkyl group, a substituted or unsubstituted C₅₋₂₀ aryl group, a substituted or unsubstituted C₅₋₂₀ aralkyl group or R¹¹ and R¹² together form a C₃₋₁₀ cycloalkyl group, all of these optionally substituted with one or more heteroatoms selected from oxygen, nitrogen and sulfur; and
R¹⁰, R¹³, R¹⁴, R¹⁵ and R¹⁶ are the same or different and each is a protecting group.

35. The process defined in claim 34, wherein the protecting group is selected from the group comprising a C₁₋₁₀ alkyl group, a C₅₋₁₀ aryl group, a C₃₋₁₀ cycloalkyl group, a C₁₋₁₀ alkylsilyl group, a C₅₋₁₀ arylsilyl group and analogs thereof substituted with one or more of a halogen, oxygen, sulfur, a nitro group, a silyl group, a thio group and a cyano group.

36. The process defined in claim 34, wherein the protecting group is selected from the group comprising a C₁₋₁₀ alkyl group, a C₅₋₁₀ aryl group, a C₃₋₁₀ cycloalkyl group and analogs thereof substituted with one or more of a halogen, oxygen, sulfur, a nitro group, a silyl group, a thio group and a cyano group.

37. The process defined in claim 33, wherein the phosphorylation moiety is 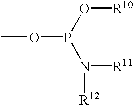
wherein R¹⁰, R¹¹ and R¹² are as defined above.

38. The process defined in claim 37, wherein R¹⁰, R¹¹ and R¹² are the same or different and each is a C₁₋₁₀ alkyl group, optionally substituted with one or more of a halogen, a nitro group, a thio group and a cyano group.

39. The process defined in claim 37, wherein R¹¹ and R¹² are the same.

40. The process defined in claim 37, wherein each of R¹¹ and R¹² is i-propyl.

41. The process defined in claim 37, wherein R¹⁰ is a C₁₋₁₀ cyanoalkyl group.

42. The process defined in claim 37, wherein R¹⁰ is a cyanoethyl group.

43. The process defined in claim 33, wherein Q¹ is selected from the group comprising a C₁₋₄₀ alkyl group, a C₅₋₄₀ aryl group, a C₅₋₄₀ alkylaryl group, a C₃₋₄₀ cycloalkyl group and analogs thereof substituted with one or more of a halogen, oxygen, sulfur, a nitro group, a silyl group, a thio group and a cyano group.

44. The process defined in claim 33, wherein Q¹ has the formula
—CH₂—CH₂—.

45. The process defined in claim 33, wherein Q¹ has the formula
—CH₂—O—CH₂—.

46. The process defined in claim 33, wherein Q¹ has the formula: 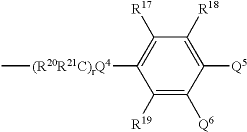
wherein: R¹⁷, R¹⁸ and R¹⁹ a re the same or different each is selected from the group comprising hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group; R²⁰ and R²¹ are the same or different and each is selected from the group comprising hydrogen, a halogen, a substituted or unsubstituted C₁-C₂₀ alkyl group, a substituted or unsubstituted C₅-C₃₀ aryl group and a substituted or unsubstituted C₅-C₄₀ alkylaryl group; Q⁴ is selected from the group consisting of —O—, —S—, —C(O)—, —S(O)₂— and —N(R)—; R is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkylaryl group; r is 0, 1 or 2; and one of Q⁵ and Q⁶ is selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group, and the other of Q⁵ and Q⁶ has the formula: 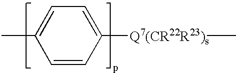
wherein p is 0 or 1, Q⁷ is selected from the group consisting of —O—, —S—, —C(O)—, —S(O)₂— and —N(R)—, R is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkylaryl group, R²² and R²³ are the same or different and are selected from the group consisting of hydrogen, a halogen, a substituted or unsubstituted C₁-C₂₀ alkyl group, a substituted or unsubstituted C₅-C₃₀ aryl group and a substituted or unsubstituted C₅-C₄₀ alkylaryl group, and s is 0, 1 or 2.

47. The process defined in claim 33, wherein:
l, m, n, o, p and q are all 1;
Q¹ is selected from
—CH₂—CH₂—
or
—CH₂—O—CH₂—
or 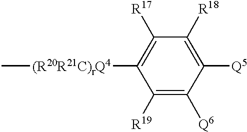
wherein: R¹⁷, R¹⁸ and R¹⁹ are the same or different each is selected from the group comprising hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group; R²⁰ and R²¹ are the same or different and each is selected from the group comprising hydrogen, a halogen, a substituted or unsubstituted C₁-C₂₀ alkyl group, a substituted or unsubstituted C₅-C₃₀ aryl group and a substituted or unsubstituted C₅-C₄₀ alkylaryl group; Q⁴ is selected from the group consisting of —O—, —S—, —C(O)—, —S(O)₂— and —N(R)—; R is selected from the group comprising hydrogen, a substituted or unsubstituted C₁—C₂₀ alkyl group, a substituted or unsubstituted C₅-C₃₀ aryl group and a substituted or unsubstituted C₅-C₄₀ alkylaryl group; r is 0, 1 or 2; and one of Q⁵ and Q⁶ is selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group, and the other of Q⁵ and Q⁶ has the formula: 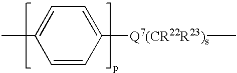
wherein p is 0 or 1, Q⁷ is selected from the group consisting of —O—, —S—, —C(O)—, —S(O)₂— and —N(R)—, R is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkylaryl group, R²² and R²³ are the same or different and are selected from the group consisting of a substituted or unsubstituted C₁-C₂₀ alkyl group, a substituted or unsubstituted C₅-C₃₀ aryl group and a substituted or unsubstituted C₅-C₄₀ alkylaryl group, and s is 0, 1 or 2;
Q² is oxygen;
Q³ is —SO₂
A¹, A², R³, R⁴, R⁵, R⁶ are all hydrogen; and
Z¹ has the following structure: 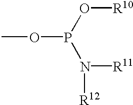
wherein R¹⁰ is 2-cyanoethyl, and R¹¹ and R¹² are each isopropyl.

48. The process defined in claim 33, wherein, prior to Step (iii), the following additional steps are conducted:
removing the terminal hydroxyl protecting group from the product of Step (ii) and then reacting the product with another compound of Formula I to produce a second derivatized support material; and
reacting the second derivatized support material with at least one nucleotide until an oligonucleotide sequence corresponding to a second oligonucleotide of interest has been synthesized.

49. The process defined in claim 48, wherein the first oligonucleotide of interest and the second oligonucleotide of interest have substantially the same sequence.

50. The process defined in claim 48, wherein the first oligonucleotide of interest and the second oligonucleotide of interest have substantially different sequences.

51. The process defined in claim 48, wherein Step (iii) comprises substantially concurrent cleavage of the first oligonucleotide of interest and the second oligonucleotide of interest.

52. The process defined in claim 48, wherein Step (iii) comprises sequential cleavage of the first oligonucleotide of interest and the second oligonucleotide of interest.

53. The process defined in claim 48, wherein the removing and reacting steps are conducted in a cyclical manner for at least two cycles to produce at least three oligonucleotides of interest.

54. The process defined in claim 53, wherein Step (iii) comprises substantially concurrent cleavage of the at least three oligonucleotides of interest.

55. The process defined in claim 53, wherein Step (iii) comprises sequential cleavage of the at least three oligonucleotides of interest.

56. A process for producing a derivatized nucleoside having having Formula Va or Formula Vb: 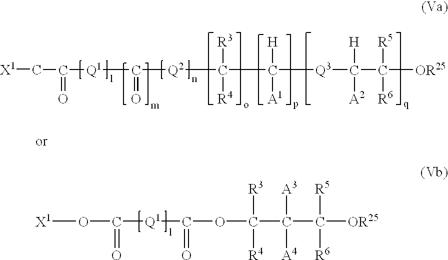
wherein:
X¹ comprises a protected nucleoside moiety selected from the following structures: 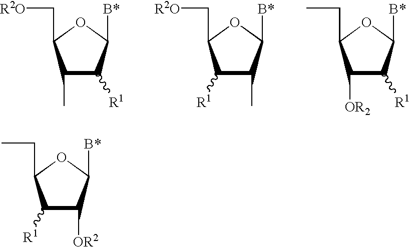
wherein:
R¹ is hydrogen, fluorine or —OR³;
R² and R³ are the same or different and each is selected from hydrogen, methyl and a protecting group; and
B* is a nucleic acid base;
Q¹ is an organic moiety;
Q² is selected from —O—, —N(H)—, —N(R⁷)— and —S—;
Q³ is selected from —S(O)₂—, —S(O)—, —C(O)—, —O—, —O—(R⁸)—O— and —R⁹—;
A¹ and A² may be the same or different and each is selected from hydrogen, halogen, a C₁₋₁₀ alkyl group, a C₅₋₁₀ aryl group, a C₃₋₁₀ cycloalkyl group, —COOR⁷, —CONH, —CONR⁷, —CN, —NO₂, —SR⁷, —S(O)R⁷, —S(O)₂R⁷, —SC(C₆H₅)₃, a C₁₋₄₀ alkylsulfonyl group, a C₅₋₁₀ aryl group, a C₁₋₁₀ alkylthio group, —Si(R⁷)₃, a C₁₋₁₀ haloalkyl group, naphthyl, 9-fluorenyl, 2-anthraquinonyl, 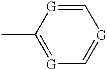
wherein G is C or N with at least one G being N, and 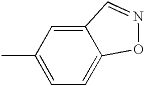
A³ and A⁴ may be the same or different and each is selected from hydrogen, halogen, a C₁₋₁₀ alkyl group, a C₅₋₁₀ aryl group, a C₃₋₁₀ cycloalkyl group and an electron withdrawing group, provided that at least one of A³ and A⁴ comprises an electron withdrawing group;
R³, R⁴, R⁵ and R⁶ are the same or different and each is selected from hydrogen, halogen, a C₁₋₁₀ alkyl group, a C₅₋₁₀ aryl group and a C₃₋₁₀ cycloalkyl group;
R⁷ is selected from a C₁₋₁₀ alkyl group, a C₅₋₁₀ aryl group and a C₃₋₁₀ cycloalkyl group;
R⁸ is a C₁₋₁₀ alkyl group or a C₅₋₁₀ aryl group;
R⁹ is a C₅₋₁₀ aryl group or —CH₂—;
l, m, n and p are independently 0 or 1;
o is an integer in the range 0-30;
q is an integer in the range 0-50; and
R²⁵ is hydrogen, a protecting group or Z¹, wherein Z¹ is a phosphorylation moiety;
the process comprising the step of reacting together compounds having Formula II and VI: 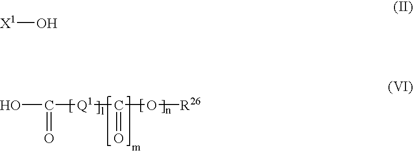
R²⁶ is hydrogen or a protecting group, with a compound having Formula VIIa (in the case where the nucleoside of Formula Va is being produced) or VIIb (in the case where the nucleoside of Formula Vb is being produced):

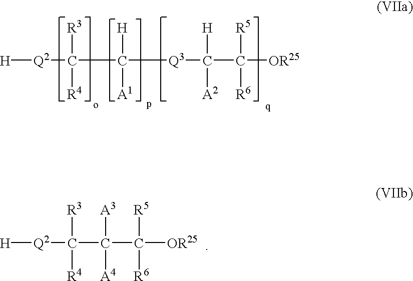

57. The process defined in claim 56, wherein:
l, o and q are independently 0 or 1;
m and n are each 1; and
o is an integer in the range 0-10.

58. The process defined in claim 56, comprising the steps of reacting compounds of Formula II and VI to produce a reaction product, and thereafter reacting the reaction product with the compound of Formula VIIa or Formula VIIb to produce the compound of Formula Va or Formula Vb.

59. The process defined in claim 56, comprising the steps of reacting compounds of Formula VI and VIIa or VI and VIIb to produce a reaction product, and thereafter reacting the reaction product with the compound of Formula II to produce the compound of Formula Va or Vb.

60. The process defined in claim 56, wherein the protecting group is selected from the group comprising a substituted or unsubstituted C₁₋₂₀ alkyl group, a substituted or unsubstituted C₅₋₃₀ aryl group, a C₃₋₁₀ cycloalkyl group, a C₅₋₄₀ alkaryl group, a C₁₋₂₀ haloalkyl group, a C₅₋₃₀ haloaryl group, a C₃₋₁₀ halocycloalkyl group, a C₁₋₂₀ nitroalkyl group, a C₅₋₂₀ nitroaryl group, a C₃-10 nitrocycloalkyl group, a C₁₋₂₀ thioalkyl group, a C₅₋₃₀ thioaryl group, a C₃₋₁₀ thiocycloalkyl group, a C₁₋₂₀ cyanoalkyl group, a C₅₋₃₀ cyanoaryl group, a C₃₋₁₀ cyanocycloalkyl group, a C₁₋₂₀ alkylsilyl group and a C₅₋₃₀ arylsilyl group.

61. The process defined in claim 56, wherein the protecting group is selected from the group comprising a C₁₋₁₀ alkyl group, a C₅₋₁₀ aryl group, a C₃₋₁₀ cycloalkyl group, a C₁₋₁₀ alkylsilyl group, a C₅₋₁₀ arylsilyl group and analogs thereof substituted with one or more of a halogen, oxygen, sulfur, a nitro group, a silyl group, a thio group and a cyano group.

62. The process defined in claim 56, comprising the further step of reacting the compound of Formula Va or Formula Vb with a compound of Formula IV
Z² (IV)
wherein Z² is a phosphorus containing precursor to Z¹ or activated phosphorylatoin moiety.

63. The process defined in claim 62, wherein Z¹ is selected from the group comprising: 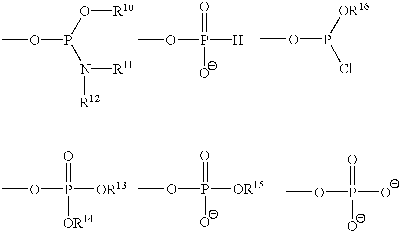
wherein:
R¹¹ and R¹² are the same or different and each may be a substituted or unsubstituted C₁₋₂₀ alkyl group, a substituted or unsubstituted C₅₋₂₀ aryl group, a substituted or unsubstituted C₅₋₂₀ aralkyl group or R¹¹ and R¹² together form a C₃₋₁₀ cycloalkyl group, all of these optionally substituted with one or more heteroatoms selected from oxygen, nitrogen and sulfur; and
and R¹⁰, R¹³, R¹⁴, R¹⁵ and R¹⁶ are the same or different and each is a protecting group.

64. The process defined in claim 62, wherein the Z′ is 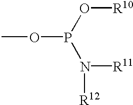
wherein R¹⁰, R¹¹ and R¹² are as defined above.

65. The process defined in claim 64, wherein R¹⁰, R¹¹ and R¹² are the same or different and each is a C₁₋₁₀ alkyl group, optionally substituted with one or more of a halogen, oxygen, sulfur, a nitro group, a silyl group, a thio group and a cyano group.

66. The process defined in claim 64, wherein R¹¹ and R¹² are the same.

67. The process defined in claim 64, wherein each of R¹¹ and R¹² is i-propyl.

68. The process defined in claim 64, wherein R¹⁰ is a C₁₋₁₀ cyanoalkyl group.

69. The process defined in claim 64, wherein R¹⁰ is a cyanoethyl group.

70. The process defined in claim 56, wherein Q¹ is selected from the group comprising a C₁₋₄₀ alkyl group, a C₅₋₄₀ aryl group, a C₅₋₄₀ alkylaryl group, a C₃₋₄₀ cycloalkyl group and analogs thereof substituted with one or more of a halogen, oxygen, sulfur, a nitro group, a silyl group, a thio group and a cyano group.

71. The process defined in claim 56, wherein:
l, m, n, o, p and q are all 1;
Q¹ is selected from
—CH₂—CH₂—
or
—CH₂—O—CH₂—
or 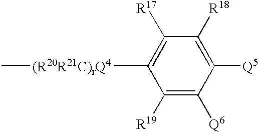
wherein: R¹⁷, R¹⁸ and R¹⁹ are the same or different each is selected from the group comprising hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group; R²⁰ and R²¹ are the same or different and each is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkylaryl group; Q⁴ is selected from the group consisting of —O—, —S—, —C(O)—, —S(O)₂— and —N(R)—; R is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkylaryl group; r is 0, 1 or 2; and one of Q⁵ and Q⁶ is selected from the group consisting of hydrogen, halide, a substituted or unsubstituted C₁-C₂₀ alkyl group, a substituted or unsubstituted C₅-C₃₀ aryl group and a substituted or unsubstituted C₅-C₄₀ alkylaryl group, and the other of Q⁵ and Q⁶ has the formula: 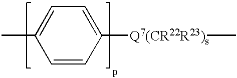
wherein p is 0 or 1, Q⁷ is selected from the group consisting of —O—, —S—, —C(O)—, —S(O)₂— and —N(R)—, R is selected from the group comprising hydrogen, a substituted or unsubstituted C₁-C₂₀ alkyl group, a substituted or unsubstituted C₅-C₃₀ aryl group and a substituted or unsubstituted C₅-C₄₀ alkylaryl group, R¹⁶ and R¹⁷ are the same or different and are selected from the group consisting of a substituted or unsubstituted C₁-C₂₀ alkyl group, a substituted or unsubstituted C₅-C₃₀ aryl group and a substituted or unsubstituted C₅-C₄₀ alkylaryl group, and s is 0, 1 or 2;
Q² is oxygen;
Q³ is —SO₂
A¹, A², R³, R⁴, R⁴, R⁵, R⁶ are all hydrogen; and
the phosphoiylatoin moiety has the following structure: 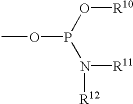
wherein R¹⁰ is 2-cyanoethyl, and R¹¹ and R¹² are each diisopropyl.

72. The process defined in claim 56, wherein R²⁵ is Z¹ and, prior to said step, the compound of Formula VIIa or Formula VIIb is first reacted with a compound of Formula IV:
Z²
wherein Z is a phosphorus containing precursor to Z¹ or an activated phosphorylating moiety, and therafter reacted sequentially with the compound of Formula (II) and the compound of Formula (VI).
